(19) United States
(12) Patent Application Publication
(10) Pub. No.: US 2025/0327801 A1
(43) Pub. Date: Oct. 23, 2025

(54) SYSTEMS AND METHODS FOR AIRBORNE ENVIRONMENTAL DETECTION AND SURVEILLANCE OF PATHOGENS WITH ELECTROCHEMICAL ANALYSIS

(71) Applicant: Washington University, St. Louis, MO (US)

(72) Inventors: John R. Cirrito, St. Louis, MO (US); Carla Yuede, St. Louis, MO (US); Rajan Chakrabarty, St. Louis, MO (US); Benjamin Sumlin, St. Louis, MO (US); Joseph Puthussery, St. Louis, MO (US)

(21) Appl. No.: 18/856,654

(22) PCT Filed: Apr. 14, 2023
(86) PCT No.: PCT/US2023/018607

§ 371 Date: Oct. 14, 2024

Related U.S. Application Data
(60) Provisional application No. 63/363,055, filed on Apr. 15, 2022.

(51) Int. Cl. G01N 33/569; G01N 33/543

(52) U.S. Cl. CPC G01N 33/56983; G01N 33/5438; G01N 33/56905; G01N 33/56911; G01N 33/56961; G01N 2333/165; G01N 2333/195; G01N 2333/37; G01N 2333/44

(57) ABSTRACT

The present disclosure is directed to an airborne detection device, method, and system for analyzing an environmental air sample and detecting airborne pathogens. The device includes an analysis vial, and a biosensor electrode. The system further includes an external sampling device.

### CROSS-REFERENCE TO RELATED APPLICATIONS

This application claims priority to U.S. Provisional Application Ser. No. 63/363,055, filed on Apr. 15, 2022, the content of which is hereby incorporated by reference in its entirety.

### STATEMENT REGARDING FEDERALLY SPONSORED RESEARCH & DEVELOPMENT

This invention was made with government support under U01AA029331 and 1RF1AG064902 awarded by the National Institutes of Health. The government has certain rights in the invention.

### FIELD OF THE DISCLOSURE

The field of the disclosure relates generally to devices, systems, and methods for surveying and detecting pathogens in the air. More specifically, the present disclosure is directed to pathogen detection and surveillance of defined air spaces, such as small and large gathering spaces.

### BACKGROUND OF THE DISCLOSURE

Coronavirus disease 2019 (COVID-19), first reported in December 2019, has afflicted 6.2 million Americans and resulted in 190,000 deaths in the United States and nearly 900,000 deaths worldwide as of early September 2020 (according to the WHO website); a roughly 3% mortality. Due to a dearth in testing and an unknown number of asymptomatic individuals, the actual number of those infected could be 6 to 24-fold higher than that reported. SARS-CoV-2 (CoV-2), the virus underlying the disease, results in a range of symptoms, in some individuals causing fever, cough, nausea, and aches. In select cases, a severe respiratory illness that impedes breathing can lead to hospitalization and death. Many individuals, however, are virtually asymptomatic but can shed virus comparable to someone that is symptomatic and remain contagious. Unfortunately, COVID-19 is likely to be prevalent well into 2021 and beyond, even if a vaccine becomes available in the beginning of the new year. Finding novel means to detect the virus, as well as create a platform to detect other pathogens, would enable us to limit the viral spread throughout the community in present and future pandemics.

The disease is caused by Severe Acute Respiratory Syndrome Coronavirus 2 (SARS-CoV-2; CoV-2) which is transmitted human-to-human either by fomites on contaminated surfaces or by aerosols. CoV-2 is transmitted person-to-person via inhalation of the virus through mucosal membranes of the nose and throat. This can occur by touching fomites on a surface, then bringing the virus into the mouth or nose via the hands. More prevalent, however, is airborne transmission. A person sheds virus in their respiratory secretions. The more forceful the respiratory fluids are expelled, the more virus is released and often over a greater distance. As such, talking expels relatively little virus whereas shouting or singing produces much more and over a larger area. Face covering and masks are designed to limit respiratory droplets escaping an infected individual, as well as limit inhalation by another individual.

The virus resides in respiratory droplets and aerosols. Large droplets carry more virus and fall to the ground faster, whereas aerosols which are smaller, carry less virus and travel a greater distance. The size of the particles is also dynamic, often changing sizes within the environment based on temperature and relative humidity (RH). A larger droplet can evaporate water to reduce its size similar to an aerosol, while an aerosol can take on water in a humid atmosphere to grow in volume. These are critical factors when considering how far a virus can travel, as well as designing means to collect and measure the virus in the air.

One major impediment to slowing the spread of CoV-2 is that asymptomatic individuals may be contagious but display minimal or no symptoms. Detecting the virus rapidly (e.g., in real-time or within seconds, minutes, or hours) could slow the spread in several dimensions. Accordingly, there is a need for rapid non-invasive tests to detect the viruses and other pathogens, even if an individual is asymptomatic, to allow for quick contact tracing and isolation.

### BRIEF DESCRIPTION OF THE DISCLOSURE

In one aspect, the present disclosure is directed to an airborne detection device for analyzing an environmental air sample and detecting airborne pathogens, the device comprising: an analysis vial; and a biosensor electrode.

In another aspect, the present disclosure is directed to a method for detecting airborne pathogens, the method comprising: transporting a liquid sample from an external sampling device to an analysis vial of an airborne detection device; adding working fluid to the analysis vial of the airborne detection device; detecting at least one pathogen; replenishing sample fluid to the external sampling device; and evacuating an analyzed sample from the analysis vial to a waste reservoir of the airborne detection device.

In yet another aspect, the present disclosure is directed to a system for detecting airborne pathogens, the system comprising: an airborne detection device; and an external sampling device.

In some embodiments, multiple pathogens are detectable simultaneously in a single test, wherein the multiple pathogens are selected from combinations of viruses, bacteria, parasites, fungi, and mold, or multiple viruses, multiple bacteria, or multiple species or strains of viruses, bacteria, parasites, fungi, or mold. In some embodiments, multiple variants of a pathogen are detectable from a single test, and wherein the multiple variants include delta and omicron variants of SARS-CoV-2. In some embodiments, multiple pathogens are detected simultaneously in a multiplex test.

### BRIEF DESCRIPTION OF THE DRAWINGS

The embodiments described herein may be better understood by referring to the following description in conjunction with the accompanying drawings.

FIG. 1A is an exemplary embodiment of SARS-CoV-2 biosensor design in accordance with the present disclosure. The CoV-2 biosensor uses square wave voltammetry to measure oxidation of tyrosine amino acids within the viral particle. Oxidation releases electrons that the sensor detects as current. The biosensor uses a nanobody attached to the surface to provide specificity and concentrate the viral particle at the electrode for measurement. The electrode is blocked with albumin to limit non-specific signal.

FIG. 1B is an exemplary embodiment of an air sampler and biosensor for continuous, real-time measurement of airborne pathogens (including CoV-2) in accordance with the present disclosure.

FIGS. 2A-2F depict an exemplary embodiment of affinity binding curves of isolated anti-SARS-CoV-2 RBD nanobodies in accordance with the present disclosure. The RBD-bound sensors were incubated with specific concentrations of purified candidate nanobodies for a set time interval to allow association (KON). The sensors were then moved to nanobody-free solution and allowed to dissociate over a time interval (KOFF). FIG. 2A shows affinity binding curves for NIH-CoVnb-109. FIG. 2B shows affinity binding curves for NIH-CoVnb-108. FIG. 2C shows affinity binding curves for NIH-CoVnb-103. FIG. 2D shows affinity binding curves for NIH-CoVnb-113. FIG. 2E shows affinity binding curves for NIH-CoVnb-112. FIG. 2F is a table showing 1:1 curve fitting used to calculate K_{D} for each nanobody.

FIG. 3A-3B depict an exemplary embodiment of NIH-CoVnb-112 specificity binding kinetics to ACE in accordance with the present disclosure. FIG. 3A shows SARS-CoV-1 RBD (blue open circles) and SARS-CoV-2 RBD (black open circles) were coated on to an ELISA plate at 10 micrograms/mL and incubated with a range of NIH-CoVnb-112 concentrations. An anti-alpaca secondary antibody was used for detection and confirms the lack of NIH-CoVnb-112 binding to SARS-CoV-1 RBD. FIG. 3B shows a CoV-2 RBD coated ELISA plate was blocked with non-specific protein and incubated with serial dilutions of each candidate anti-SARS-CoV-2 RBD nanobody. Biotinylated-ACE2 was added to each well and allowed to bind to unoccupied RBD. Unoccupied RBD allows for a positive reaction signal which is suppressed in the presence of bound competitive nanobody.

FIG. 4 is an exemplary embodiment of NIH-CoVnb-112 CoV-2 neutralization in accordance with the present disclosure. FRNA50 assays were performed to determine neutralizing activity of anti-SARS-CoV-2 nanobody. Vero E6 cells (8×10⁴ cells per well) were plated on 96-well Operetta-compatible plates (PerkinElmer). Live CoV-2 virus+NIH-CoV2nb-112 dilutions were added to the 96-well plates and incubated for 24 hours. Cells were fixed and stained with a SARS-CoV-2 anti-spike antibody, followed by goat anti-rabbit IgG Alexa Fluor 594. Images were taken on a Operetta High Content Imager.

FIG. 5A-5B depict an exemplary embodiment of Aβ oligomer standard curves in accordance with the present disclosure. Example Aβ oligomer standard curve using synthetic Aβ dimer on the MIE (FIG. 5A) and ELISA (FIG. 5B) with lower limit of detections of 200 attograms/ml and 32 pg/ml, respectively.

FIG. 6A-6B depict an exemplary embodiment of SARS-CoV-2 Spike Protein Biosensor in accordance with the present disclosure. MIE biosensor coupled with 100 μg/ml of NIH-CoVnb-112. Square wave voltammetry was used to detect CoV-2 RBD spike protein ranging from 2 fg/ml to 2 ng/ml (r2=0.9088). The study was replicated 8 times; showing representative sensitivity curve (FIG. 6A) and oxidation peaks (FIG. 6B).

FIG. 7 is an exemplary embodiment of aerosol dynamics and residence time in accordance with the present disclosure. Trajectories of a 10 μm (green) and 100 μm (blue) particle emitted with an initial velocity of release typical of a sneezing person. Perforated lines depict no evaporation, while solid lines represent evaporation for the particle upon release to the atmosphere. The life time of a 10 μm particle increases from 8.3 min (no evaporation) to 12 hours (with evaporation); and for a 100 μm particle from 4.9 s to 39.4 s.

FIG. 8 is an exemplary embodiment of portable PalmSens4 potentiostat in accordance with the present disclosure. PalmSens4 potentiostat with graphite screen printed electrode. The device can be run connected to a computer or through Bluetooth on a smartphone.

FIG. 9 is an exemplary embodiment of a schematic diagram of the experimental set up for virus aerosol generation, environmental processing, sampling, and characterization in accordance with the present disclosure.

FIG. 10 is an exemplary embodiment of an atmospheric aerosol generation laboratory setup in accordance with the present disclosure. Particle/gas stream (ambient, aerosolized, combusted, emitted, denuded or un-denuded) is sent through a cyclone, DMA, or teflon filter for sample preparation. Variable humidity, O₃ (plus option for addition of, or sample substitution by VOCs, SO₂, NOₓ, NH₃, or condensable organic aerosol) input is combined with sample in a Potential Aerosol Mass (PAM) reaction chamber to mimic atmospheric oxidation and photochemistry processes. Oxidant levels are adjusted by varying input O₃, H₂O, lamp voltage, and lamp type. Default lamp is low pressure Hg w/strong 254 nm wavelength and option for 185 nm w/removal of quartz sleeve.

FIG. 11 is an exemplary embodiment of a laboratory aerosolization test chamber setup in accordance with the present disclosure. The chamber if filled by aerosolizing test particle/virus at fixed nebulization rate. The concentration inside the chamber is continuously monitored using a particle counter. Multiple PILS devices can be placed inside the chamber for parallel sample collection.

FIG. 12A is an exemplary embodiment of an airborne detection system in accordance with the present disclosure.

FIG. 12B is an exemplary embodiment of an external view of the desktop airborne detection device in accordance with the present disclosure.

FIG. 12C is an exemplary embodiment of an external view of the duct air sampling airborne detection device in accordance with the present disclosure.

FIG. 13 is an exemplary embodiment of a method for detecting airborne pathogens in accordance with the present disclosure.

FIG. 14A is an exemplary embodiment of the wet cyclone PILS dimensions in accordance with the present disclosure.

FIG. 14B is an exemplary embodiment of the wet cyclone PILS boundary conditions assumed for numerical simulations in accordance with the present disclosure.

FIG. 15 is an exemplary embodiment of the boundary condition assumed for the numerical modeling simulations in accordance with the present disclosure.

FIG. 16 is an exemplary embodiment of the size-specific particle recovery in the wet cyclone based on numerical modeling simulations in accordance with the present disclosure.

FIG. 17 is an exemplary embodiment of the Chamber experiments comparing the wet cyclone PILS performance with the commercial PILS (BioSampler® and LSS) for three aerosolized virus concentration levels: <500 copies/m³ (low), 500-10,000 copies/m³ (medium), and >10,000 copies/m³ (high) in accordance with the present disclosure.

FIG. 18 is an exemplary embodiment of the PCR Ct value (inverted y-axis) of indoor air samples collected using the wet cyclone in apartments with SARS-CoV-2 positive patients and control room in accordance with the present disclosure.

FIG. 19A is an exemplary embodiment of the laboratory characterization of the pAQ monitor to calculate the SARS-CoV-2 variant-specific LoD in accordance with the present disclosure.

FIG. 19B is an exemplary embodiment of the proof of concept box plot data showing the pAQ monitor oxidation current while sampling aerosolized inactivated CoV-2 in accordance with the present disclosure.

FIG. 20 is an exemplary embodiment of the virus aerosolization experiment set-up to determine the pAQ sensitivity in accordance with the present disclosure.

### DETAILED DESCRIPTION OF THE DISCLOSURE

The present disclosure describes an environmental sensor that detects aerosolized virus within a given space (such as any indoor or enclosed space, including large spaces or other spaces having a potentially shared airspace) to determine if viral particles (such as aerosolized CoV-2) are present. Also described is an electrochemical, antibody-based biosensor to detect inactivated viral particles of at least one respiratory virus (or several respiratory viruses) alternatively or additional to SARS-CoV-2 virus particles. In some embodiments, the biosensor detects at least one bacterial genus or species. In other embodiments, the device detects at least one parasite, fungi, or mold genus or species.

Viral particles detectable by the disclosed biosensor include, but are not limited to, viruses associated with Chikungunya, Cholera, Crimean-Congo hemorrhagic fever, Ebola virus disease, Hendra virus infection, Influenza (pandemic, seasonal, zoonotic), Lassa fever, Marburg virus disease, Meningitis, MERS-CoV, Monkeypox, Nipah virus infection, Novel coronavirus (2019-nCoV), Plague, Rift Valley fever, SARS, Smallpox, Tularaemia, Yellow fever, Zika virus disease, Ebola and Marburg virus (Filoviridae); Ross River virus, chikungunya virus, Sindbis virus, eastern equine encephalitis virus (Togaviridae, Alphavirus), vesicular stomatitis virus (Rhabdoviridae, Vesiculovirus), Amapari virus, Pichinde virus, Tacaribe virus, Junin virus, Machupo virus (Arenaviridae, Mammarenavirus), West Nile virus, dengue virus, yellow fever virus (Flaviviridae, Flavivirus); human immunodeficiency virus type 1 (Retroviridae, Lentivirus); Moloney murine leukemia virus (Retroviridae, Gammaretrovirus); influenza A virus (Orthomyxoviridae); respiratory syncytial virus (Paramyxoviridae, Pneumovirinae, Pneumovirus); vaccinia virus (Poxviridae, Chordopoxvirinae, Orthopoxvirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus); Autographa californica nucleopolyhedrovirus (Baculoviridae, Alphabaculoviridae) (an insect virus); Ebola and Marburg virus (Filoviridae); Semliki Forest virus, Ross River virus, chikungunya virus, O'nyong-nyong virus, Sindbis virus, eastern/western/Venezuelan equine encephalitis virus (Togaviridae, Alphavirus); rubella (German measles) virus (Togaviridae, Rubivirus); rabies virus, Lagos bat virus, Mokola virus (Rhabdoviridae, Lyssavirus); Amapari virus, Pichinde virus, Tacaribe virus, Junin virus, Machupo virus, Guanarito virus, Sabia virus, Lassa virus (Arenaviridae, Mammarenavirus); West Nile virus, dengue virus, yellow fever virus, Zika virus, Japanese encephalitis virus, St. Louis encephalitis virus, tick-borne encephalitis virus, Omsk hemorrhagic fever virus, Kyasanur Forest virus (Flaviviridae, Flavivirus); human hepatitis C virus (Flaviviridae, Hepacivirus); human immunodeficiency virus type 1 (Retroviridae, Lentivirus); influenza A/B virus (Orthomyxoviridae, the common ‘flu’ virus); respiratory syncytial virus (Paramyxoviridae, Pneumovirinae, Pneumovirus); Hendra virus, Nipah virus (Paramyxoviridae, Paramyxovirinae, Henipavirus); measles virus (Paramyxoviridae, Paramyxovirinae, Morbillivirus); Variola major (smallpox) virus (Poxviridae, Chordopoxvirinae, Orthopoxvirus); human hepatitis B virus (Hepadnaviridae, Orthohepadnavirus); hepatitis delta virus (hepatitis D virus) (unassigned Family, Deltavirus); herpes simplex virus type 1, herpes simplex virus type 2 (Herpesviridae, Alphaherpesvirinae, Simplexvirus); human cytomegalovirus (Herpesviridae, Betaherpesvirinae, Cytomegalovirus), Adeno-associated virus Dependovirus, Parvoviridae Aichi virus Kobuvirus, Picornaviridae Australian bat lyssavirus, Rhabdoviridae BK polyomavirus, Polyomaviridae Banna virus Seadornavirus, Reoviridae Barmah forest virus Alphavirus, Togaviridae Bunyamwera virus Orthobunyavirus, Bunyaviridae Bunyavirus La Crosse Orthobunyavirus, Bunyaviridae Bunyavirus snowshoe hare Orthobunyavirus, Bunyaviridae Cercopithecine herpesvirus Lymphocryptovirus, Herpesviridae Chandipura virus Vesiculovirus, Rhabdoviridae Chikungunya virus Alphavirus, Togaviridae Cosavirus A Cosavirus, Picornaviridae Cowpox virus Orthopoxvirus, Poxviridae Coxsackievirus Enterovirus, Picornaviridae Crimean-Congo hemorrhagic Nairovirus, Bunyaviridae fever virus Dengue virus Flavivirus, Flaviviridae Dhori virus Thogotovirus, Orthomyxoviridae Dugbe virus Nairovirus, Bunyaviridae Duvenhage virus Lyssavirus, Rhabdoviridae Eastern equine encephalitis virus Alphavirus, Togaviridae Ebolavirus, Filoviridae Echovirus Enterovirus, Picornaviridae Encephalomyocarditis virus Cardiovirus, Picornaviridae Epstein-Barr virus Lymphocryptovirus, Herpesviridae European bat lyssavirus, Rhabdovirus GB virus C/Hepatitis G virus Pegivirus, Flaviviridae Hantaan virus Hantavirus, Bunyaviridae Hendra virus Henipavirus, paramyxoviridae Hepatitis A virus Hepatovirus, picornaviridae Hepatitis B virus Orthohepadnavirus, Hepadnaviridae Hepatitis C virus Hepacivirus, Flaviviridae Hepatitis E virus Hepevirus, Unassigned Hepatitis delta virus Deltavirus, Unassigned Horsepox virus Orthopoxvirus, Poxviridae Human adenovirus Mastadenovirus, Adenoviridae Human astrovirus Mamastrovirus, Astroviridae Human coronavirus Alphacoronavirus, Coronaviridae Human cytomegalovirus, Herpesviridae Human enterovirus 68, 70 Enterovirus, Picornaviridae Human herpesvirus 1 Simplexvirus, Herpesviridae Human herpesvirus 2 Simplexvirus, Herpesviridae Human herpesvirus 6 Roseolovirus, Herpesviridae Human herpesvirus 7 Roseolovirus, Herpesviridae Human herpesvirus 8 Rhadinovirus, Herpesviridae Human immunodeficiency virus Lentivirus, Retroviridae Human papillomavirus 1 Mupapillomavirus, Papillomaviridae Human papillomavirus 2 Alphapapillomavirus, Papillomaviridae Human papillomavirus 16, 18 Alphapapillomavirus, Papillomaviridae Human parainfluenza Respirovirus, Paramyxoviridae Human parvovirus B19 Erythrovirus, Parvoviridae Human respiratory syncytial virus Orthopneumovirus, Pneumoviridae Human rhinovirus Enterovirus, Picornaviridae Human SARS coronavirus Betacoronavirus, Coronaviridae Human spumaretrovirus Spumavirus, Retroviridae Human T-lymphotropic virus Deltaretrovirus, Retroviridae Human torovirus, Coronaviridae Influenza A virus Influenzavirus A, Orthomyxoviridae Influenza B virus Influenzavirus B, Orthomyxoviridae Influenza C virus Influenzavirus C, Orthomyxoviridae Isfahan virus Vesiculovirus, Rhabdoviridae JC polyomavirus, Polyomaviridae Japanese encephalitis virus Flavivirus, Flaviviridae Junin arenavirus, Arenaviridae KI Polyomavirus, Polyomaviridae Kunjin virus Flavivirus, Flaviviridae Lagos bat virus Lyssavirus, Rhabdoviridae Lake Victoria marburgvirus Marburgvirus, Filoviridae Langat virus Flavivirus, Flaviviridae Lassa virus Arenavirus, Arenaviridae Lordsdale virus Norovirus, Caliciviridae Louping ill virus Flavivirus, Flaviviridae Lymphocytic choriomeningitis Arenavirus, Arenaviridae virus Machupo virus Arenavirus, Arenaviridae Mayaro virus Alphavirus, Togaviridae MERS coronavirus Betacoronavirus, Coronaviridae Measles virus Morbilivirus, Paramyxoviridae Mengo encephalomyocarditis virus Cardiovirus, Picornaviridae Merkel cell polyomavirus, Polyomaviridae Mokola virus Lyssavirus, Rhabdoviridae Molluscum contagiosum virus Molluscipoxvirus, Poxviridae Monkeypox virus Orthopoxvirus, Poxviridae Mumps virus Rubulavirus, Paramyxoviridae Murray valley encephalitis virus Flavivirus, Flaviviridae New York virus Hantavirus, Bunyavirus Nipah virus Henipavirus, Paramyxoviridae Norwalk virus Norovirus, Caliciviridae O'nyong-nyong virus Alphavirus, Togaviridae Orf virus Parapoxvirus, Poxviridae Oropouche virus Orthobunyavirus, Bunyaviridae Pichinde virus Arenavirus, Arenaviridae Poliovirus Enterovirus, Picornaviridae Punta toro phlebovirus, Bunyaviridae Puumala virus Hantavirus, Bunyavirus Rabies virus Lyssavirus, Rhabdoviridae Rift valley fever virus Phlebovirus, Bunyaviridae Rosavirus A Rosavirus, Picornaviridae Ross river virus Alphavirus, Togaviridae Rotavirus A Rotavirus, Reoviridae Rotavirus B Rotavirus, Reoviridae Rotavirus C Rotavirus, Reoviridae Rubella virus Rubivirus, Togaviridae Sagiyama virus Alphavirus, Togaviridae Salivirus A Salivirus, Picornaviridae Sandfly fever sicilian virus Phlebovirus, Bunyaviridae Sapporo virus Sapovirus, Caliciviridae Semliki forest virus Alphavirus, Togaviridae Seoul virus Hantavirus, Bunyavirus Simian foamy virus Spumavirus, Retroviridae Simian virus 5 Rubulavirus, Paramyxoviridae Sindbis virus Alphavirus, Togaviridae Southampton virus Norovirus, Caliciviridae St. louis encephalitis virus Flavivirus, Flaviviridae Tick-borne powassan virus Flavivirus, Flaviviridae Torque teno virus Alphatorquevirus, Anelloviridae Toscana virus Phlebovirus, Bunyaviridae Uukuniemi virus Phlebovirus, Bunyaviridae Vaccinia virus Orthopoxvirus, Poxviridae Varicella-zoster virus Varicellovirus, Herpesviridae Variola virus Orthopoxvirus, Poxviridae Venezuelan equine encephalitis Alphavirus, Togaviridae virus Vesicular stomatitis virus Vesiculovirus, Rhabdoviridae Western equine encephalitis virus Alphavirus, Togaviridae WU polyomavirus, Polyomaviridae West Nile virus Flavivirus, Flaviviridae Yaba monkey tumor virus Orthopoxvirus, Poxviridae Yaba-like disease virus Orthopoxvirus, Poxviridae Yellow fever virus Flavivirus, Flaviviridae Zika virus Flavivirus, and Flaviviridae.

Bacterial genera and species detectable by the disclosed biosensor include, but are not limited to, bacteria associated with Xanthomonas, Pseudomonas, Salmonella, Shigella, Chlamydia, Helicobacter, Yersinia, Bordatella, Pseudomonas, Neisseria, Vibrio, Haemophilus, Mycoplasma, Streptomyces, Treponema, Coxiella, Ehrlichia, Brucella, Streptobacillus, Fusospirocheta, Spirillum, Ureaplasma, Spirochaeta, Mycoplasma, Actinomycetes, Borrelia, Bacteroides, Trichomoras, Branhamella, Pasteurella, Clostridium, Corynebacterium, Listeria, Bacillus, Erysipelothrix, Rhodococcus, Escherichia, Klebsiella, Pseudomanas, Enterobacter, Serratia, Staphylococcus, Streptococcus, Legionella, Mycobacterium, Proteus, Campylobacter, Enterococcus, Acinetobacter, Morganella, Moraxella, Citrobacter, Rickettsia, Rochlimeae, as well as bacterial species such as: P. aeruginosa; E. coli, P. cepacia, S. epidermis, E. faecalis, S. pneumonias, S. aureus, N meningitidis, S. pyogenes, Pasteurella multocida, Treponema pallidum, and P. mirabilis.Gram-negative bacterial genera and species detectable by the disclosed biosensor include, but are not limited to, Escherichia spp., Shigella spp., Salmonella spp., Campylobacter spp., Neisseria spp., Haemophilus spp., Aeromonas spp., Francisella spp., Yersinia spp., Klebsiella spp., Bordetella spp., Legionella spp., Corynebacteria spp., Citrobacter spp., Chlamydia spp., Brucella spp., Pseudomonas spp., Helicobacter spp. and Vibrio spp. Gram-negative bacterial genera and species detectable by the disclosed biosensor include, but are not limited to, Salmonella, E. coli, Yersinia pestis, Klebsiella and Shigella, Proteus, Enterobacter, Serratia, and Citrobacter.

Fungi detectable by the disclosed biosensor include, but are not limited to, fungi associated with Cryptococcus neoformans; Blastomyces dermatitidis; Aiellomyces dermatitidis; Histoplasma capsulatum; Coccidioides immitis; Candida species, including C. albicans, C. tropicalis, C. parapsilosis, C. guilliermondii and C. krusei, Aspergillus species, including A. fumigatus, A. flavus and A. niger, Rhizopus species; Rhizomucor species; Cunninghammella species; Apophysomyces species, including A. saksenaea, A. mucor and A. absidia; Sporothrix schenckii, Paracoccidioides brasiliensis; Pseudallescheria boydii, Torulopsis glabrata; Trichophyton species, Microsporum species and Dermatophyres species, as well as any other yeast or fungus now known or later identified to be pathogenic.

Parasites detectable by the disclosed biosensor include, but are not limited to, parasites associated with Anaplocephala, Ancylostoma, Necator, Ascaris, Brugia, Bunostomum, Capillaria, Chabertia, Cooperia, Cyathostomum, Cylicocyclus, Cylicodontophorus, Cylicostephanus, Craterostomum, Dictyocaulus, Dipetalonema, Dipylidium, Dracunculus, Echinococcus, Enterobius, Fasciola, Filaroides, Habronema, Haemonchus, Metastrongylus, Moniezia, Nematodirus, Nippostrongylus, Oesophagostomum, Onchocerca, Ostertagia, Oxyuris, Parascaris, Schistosoma, Strongylus, Taenia, Toxocara, Strongyloides, Toxascaris, Trichinella, Trichuris, Trichostrongylus, Triodontophorus, Uncinaria, Wuchereria, Leishmaniasis disease, human African trypanosomiasis disease, Chagas disease, antigens derived from members of the Apicomplexa phylum such as, for example, Babesia, Toxoplasma, Plasmodium, Eimeria, Isospora, Atoxoplasma, Cystoisospora, Hammondia, Besniotia, Sarcocystis, Frenkelia, Haemoproteus, Leucocytozoon, Theileria, Perkinsus and Gregarina spp.; Pneumocystis carinii; members of the Microspora phylum such as, for example, Nosema, Enterocytozoon, Encephalitozoon, Septata, Mrazekia, Amblyospora, Ameson, Glugea, Pleistophora and Microsporidium spp.; and members of the Ascetospora phylum such as, for example, Haplosporidium spp., as well as species including Plasmodium falciparum, P. vivax, P. ovale, P. malaria; Toxoplasma gondii; Leishmania mexicana, L. tropica, L. major, L. aethiopica, L. donovani, Trypanosoma cruzi, T brucei, Schistosoma mansoni, S. haematobium, S. japonium; Trichinella spiralis; Wuchereria bancrofti; Brugia malayli; Entamoeba histolytica; Enterobius vermiculoarus; Taenia solium, T saginata, Trichomonas vaginitis, T hominis, T tenax; Giardia lamblia; Cryptosporidium parvum; Pneumocytis carinii, Babesia bovis, B. divergens, B. microti, Isospora belli, L. hominis; Dientamoeba fragilis; Onchocerca volvulus; Ascaris lumbricoides; Necator americans; Ancylostoma duodenale; Strongyloides stercoralis; Capillaria phihppinensis; Angiostrongylus cantonensis; Hymenolepis nana; Diphyllobothrium latum; Echinococcus granulosus, E. multilocularis; Paragonimus westermani, P. caliensis; Chlonorchis sinensis; Opisthorchis felineas, G. Viverini, Fasciola hepatica, Sarcoptes scabiei, Pediculus humanus; Phthirlus pubis; and Dermatobia hominis, as well as any other parasite now known or later identified to be pathogenic.

Additional pathogens detectable by the disclosed biosensor include, but are not limited to, Coronaviridae (e.g. MERS, SARS-CoV-2), Bunyavirales (e.g. Lassa, Junin, Rift Valley Fever Virus, Andes, Sin Nombre, LaCrosse, California Encephalitis, Crimean Congo Hemorrhagic Fever), Filoviruses (e.g. Ebola, Marburg), Flaviviruses (e.g. Dengue, Zika, West Nile), Paramyxoviridae (e.g. Nipah, Hendra), Picornaviridae (e.g. EV-D68, EV-A71), Togaviridae (e.g. Chikungunya, EEE, VEE, WEE), Bacillus anthracis (including genotypic resistance markers), Yersinia pestis (including genotypic resistance markers), Francisella tularensis (including genotypic resistance markers), Burkholderia spp. (including genotypic resistance markers), Botulinum toxin (including identifying and distinguishing relevant serotypes), ESKAPE pathogens including genotypic resistance markers (e.g., Enterococcus faecium, Staphylococcus aureus, Klebsiella pneumonia, Acinetobacter baumannii, Pseudomonas aeruginosa, Enterobacter spp), Lassa virus, Nipah virus, Rift Valley Fever virus, Enterovirus D68 virus, Candida auris, Coccidioides sp., and novel coronaviruses.

Aspects of the present disclosure are provided by the subject matter of the following clauses:
1. An airborne detection device for analyzing an environmental air sample and detecting airborne pathogens, the device comprising:
   an analysis vial; and
   a biosensor electrode.
2. The device of the preceding clause, wherein the device is configured to be used to detect multiple pathogens simultaneously in a single test.
3. The device of any preceding clause, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.
4. The device of any preceding clause, wherein the device is used to test for multiple variants of a pathogen from a single test.
5. The device of any preceding clause, wherein the multiple variants of a pathogen include delta and omicron variants of SARS-CoV-2.
6. The device of any preceding clause, wherein the device is configured to be used in multiplex tests.
7. The device of any preceding clause, wherein in the multiplex tests, multiple pathogens are detected simultaneously.
8. A method for detecting airborne pathogens, the method comprising:
   transporting a liquid sample from an external sampling device to an analysis vial of an airborne detection device;
   adding working fluid to the analysis vial of the airborne detection device;
   detecting at least one pathogen;
   replenishing sample fluid to the external sampling device; and evacuating an analyzed sample from the analysis vial to a waste reservoir of the airborne detection device.
9. The method of the preceding clause, wherein detecting at least one pathogen comprises detecting multiple pathogens simultaneously in a single test.
10. The method of any preceding clause, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.
11. The method of any preceding clause, wherein detecting at least one pathogen comprises detecting multiple variants of a pathogen from a single test.
12. The method of any preceding clause, wherein the multiple variants of a pathogen include delta and omicron variants of SARS-CoV-2.
13. The method of any preceding clause, wherein detecting at least one pathogen comprises multiplex detection, wherein multiple pathogens are detected simultaneously.
14. A system for detecting airborne pathogens, the system comprising:
   an airborne detection device; and
   an external sampling device.
15. The system of the preceding clause, wherein the system is used to detect multiple pathogens simultaneously in a single test.
16. The system of any preceding clause, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.
17. The system of any preceding clause, wherein the system is used to test for multiple variants of a pathogen from a single test.
18. The system of any preceding clause, wherein the multiple variants of a pathogen include delta and omicron variants of SARS-CoV-2.
19. The system of any preceding clause, wherein the system is configured to be used in multiplex tests.
20. The system of any preceding clause, wherein in the multiplex tests, multiple pathogens are detected simultaneously.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever.

Immuno-based biosensor and environmental detection of airborne pathogens. In exemplary embodiments, the biosensor will be deployed in an environmental detector for rapid (e.g., real-time or near real-time), continuous measurement of sampled air. The environmental biosensor of the present disclosure for detecting target organisms is surprisingly and unexpectedly based on an ultra-sensitive electrochemical technology used in vivo (e.g., brain, tissue, interstitial fluid, etc.) for Alzheimer's disease research for detecting macromolecular targets.

An initial micro-immunoelectrode (MIE) biosensor was developed to detect amyloid-β (Aβ) peptide in the setting of Alzheimer's disease. The electrochemical sensor uses voltammetry to measure oxidation of tyrosine amino acids within a protein. Oxidation is the release of electrons that the carbon fiber electrode detects as a change in current. The amount of current is proportional to the amount of protein present. The biosensor uses an antibody covalently attached to the surface to provide specificity and concentrate the protein at the electrode for measurement.

The biosensor of the present disclosure is based on a similar design as the MIE. Some embodiments herein describe a CoV-2 nanobody (raised in llama) with 5nM affinity for the SARS-CoV-2 repeat binding domain (RBD) of the spike protein and very high selectivity over the CoV-1 spike protein. The present disclosure demonstrates that the CoV-2 biosensor has an initial sensitivity of 2 fg/ml. In contrast, conventional antigen tests for CoV-2 are sensitive to the low pg/ml range. Development of the environmental sensor, biosensor, and methods disclosed herein included mimicking real-world environmental conditions, especially in the context of atmospheric aerosols, necessary for testing and optimizing the biosensor's performance for field deployment.

As disclosed herein, an immuno-based electrochemical biosensor provides real-time and continuous measures of CoV-2 aerosols for use in airborne environmental detection and diagnostics. In some embodiments, the sensor targets CoV-2. In other embodiments, design and methodology is adapted to numerous pathogens present in the air or from respiration. The airborne detector described herein monitors gathering spaces for environmental risks to flag for evacuation and/or enhanced disinfection.

In an exemplary embodiment, a CoV-2 biosensor has reasonable sensitivity for recombinant spike protein, and is adaptable depending on viral (pathogen) particles, antibody type, longevity, concentration, and orientation on the electrode surface. In some embodiments, the biosensor is applicable for inactivated CoV-2 viral particle detection. In these embodiments, specificity controls include surface proteins and viral particles of other viruses. Depending upon the embodiment, electrode design is optimized for the size and type of material having the largest impact on specificity and oxidation properties.

Airborne transmission of CoV-2 is caused by the dissemination of droplet nuclei (aerosols) that remain infectious when suspended in air over long distances and time. Guided by observational studies of CoV-2 and other infectious viruses, the biosensor performance metrics of the present disclosure are systematically evaluated with respect to sensitivity, detection limits, and longevity, and aerosolized viral particles are subjected to relevant environmental parameters, including relative humidity, temperature, and atmospheric residence time. The biosensor's performance also includes testing under conditions mimicking real-world indoor and urban atmospheres wherein aerosolized virus droplets are mixed with particulate matter pollutants, such as volatile organics, dust and soot. In some embodiments, an environmental sensor device sampling a given air space detects CoV-2 in real-time or near real-time over a period of at least about 12 hours to about 24 hours.

Diagnostic testing. As noted above, increased and improved ability to test for pathogens, including CoV-2, is needed. While detection and diagnosis of both symptomatic and asymptomatic individuals is needed to inform individual isolations and/or quarantines and to reduce community spread, an equally important, but underdeveloped measure is to monitor a gathering area in real-time (or near real-time) for airborne virus that could result in the shutdown of a space or warrant intense disinfection of the area. In some embodiments described herein, an airborne pathogen sensor is used in conjunction with an aerosol disinfectant mister for immediate cleansing to limit spread of the detected virus and/or pathogen.

Conventional tests for CoV-2 generally detect RNA, antigen (the virus itself), or antibodies. Each of these tests vary in sensitivity/specificity, length of analysis time, or stage of disease. RNA and antigen testing are useful to detecting current virus, whereas antibody tests identify past infection. RT-PCR tests to detect RNA vary in response time from hours to days under best scenarios, while antibody tests generally take 1-2 days for results. Antigen tests tend to be the fastest, with current saliva or nasal swabs results returned in as little as 5 minutes using a portable sensor (e.g. Abbott COVID-19 ID NOW Test). However, blood tests require trained personnel to withdraw blood and saliva tests, while fast and non-invasive, leave biological material remaining that must be disposed of safely.

Sensitivity/specificity of conventional COVID-19 tests are highly variable. RT-PCR tests tend to be the most sensitive with up to 97.4% accuracy in a clinical setting. In contrast, some serological antibody tests are less than 50% specific, making those tests almost trivial in a clinical setting. In a meta-analysis, antigen tests vary widely, with sensitivity up to 94%; however the average was 56.2% sensitivity (true positive) with 99.5% specificity (true negative). While the advantage of conventional antigen tests is shorter result time, there is still room for significant improvements for diagnostics.

In addition to personal diagnostic value of these tests, sensors still need to be developed for airborne environmental detection. Available tests for environmental virus generally includes wipe tests of surfaces or single use, repeated air sample measurements. There are no currently available tests that provide user-free, continual measures of airborne CoV-2 and/or other pathogens. The disclosed systems, device, and methods for detection of airborne environmental pathogens (including CoV-2) in real-time enable public places to be monitored to guard against outbreaks or “super-spreader” events for public safety.

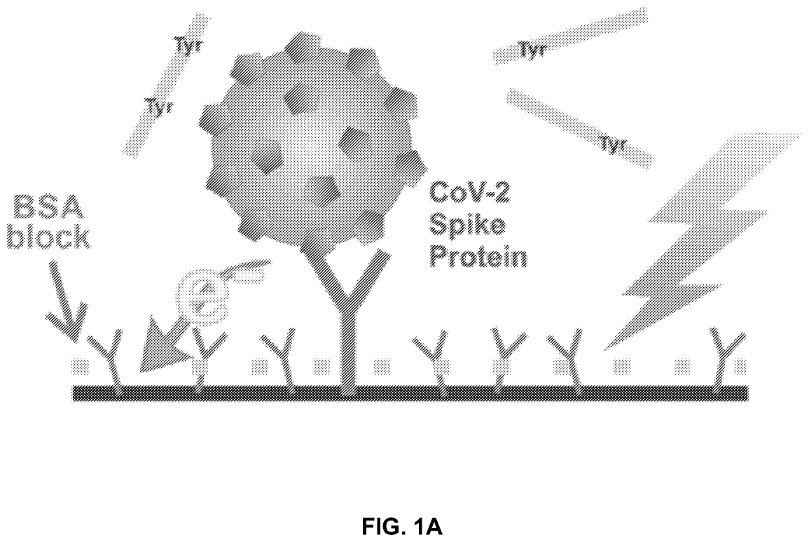

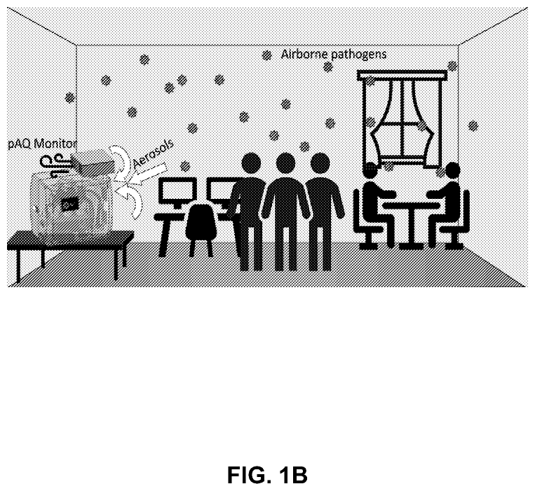

Immuno-biosensor for aerosolized and airborne detection. Disclosed herein is an ultra-sensitive immuno-based electrochemical biosensor to detect pathogens, and in exemplary embodiments, the spike protein on the surface of CoV-2. The CoV-2 biosensor (FIG. 1 (A-B)) disclosed herein is applicable for detecting airborne viruses (and/or pathogens) using an environmental sampling and detection system that can be applied to a large area, such as an airport, hospital, conference center, or school setting. Depending upon the embodiment, a specifically optimized biosensor is implemented to account for conditions of deployment and longevity of sampling and surveillance. Depending upon the embodiment, the collection platform can be modified to detect other pathogens and/or combinations of pathogens.

Innovation of the systems, methods, and devices disclosed herein is based at least in part on the immuno-based biosensor, the nanobody to provide specificity, and/or the sample collection and processing of aerosolized pathogen particles (e.g., CoV-2 viral particles) to real-world environmental conditions prior to testing on the biosensor.

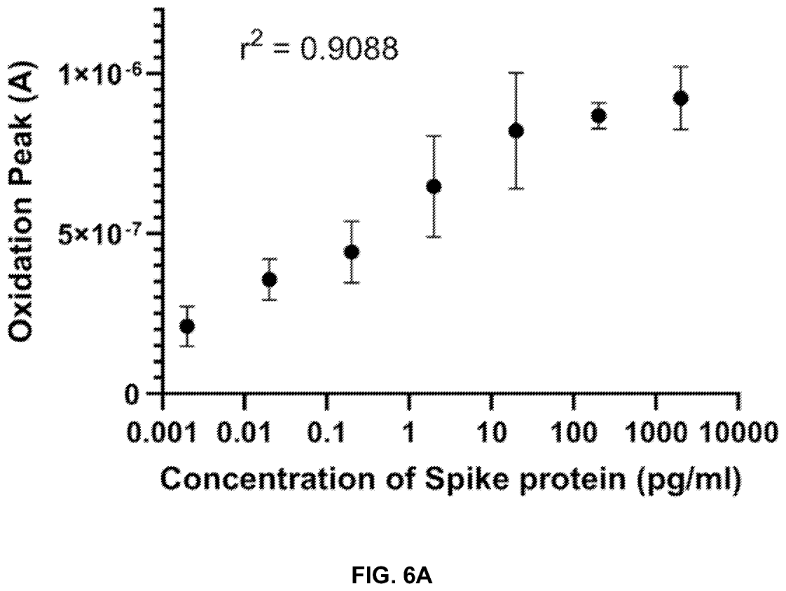

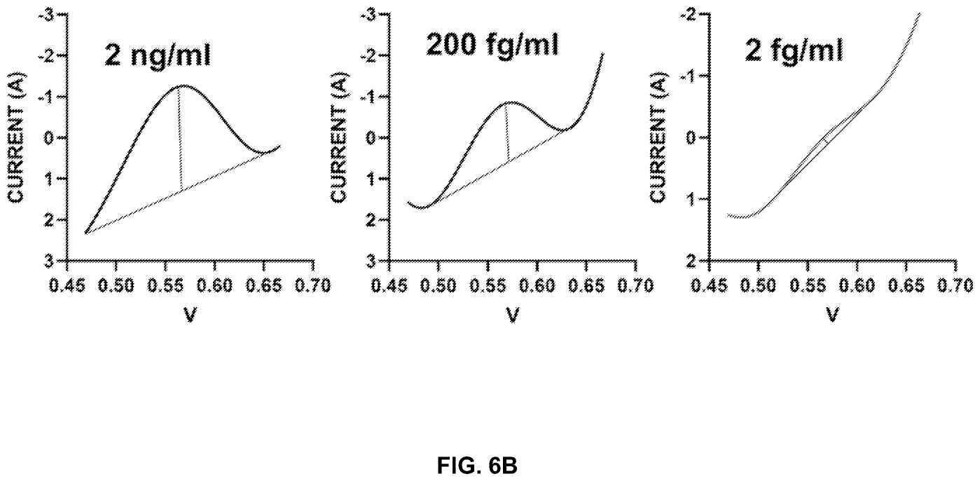

CoV-2 Biosensor. Micro-immunoelectrode (MIE) technology as disclosed herein uses square wave voltammetry to measure oxidation of tyrosine amino acids in specific proteins. In embodiments of the CoV-2 biosensor, the biosensor sensitivity has been observed down to 2 fg/ml of CoV-2 spike RBD protein (FIG. 6), which in contrast to conventional CoV-2 immunosorbent assays in the low pg/ml range. In some embodiments, the biosensor uses recombinant spike protein. In other embodiments, the biosensor uses CoV-2 viral particles.

As disclosed herein, specificity for a target is based on an antibody covalently attached to the electrode surface. Oxidation of the CoV-2 spike protein bound to the antibody was measured as a direct measure that protein is present. Importantly, tyrosine oxidation is irreversible, meaning the protein bound to the antibody on the surface of the electrode will only be measured once. This is in contrast to many conventional electrochemical sensors that measure impedance at the electrode surface; essentially measuring the binding event instead of the actual protein. Impedance measures can be fraught with specificity issues since non-specific proteins or molecules can deposit on the surface of the electrode and also produce a signal, often referred to as “fouling”.

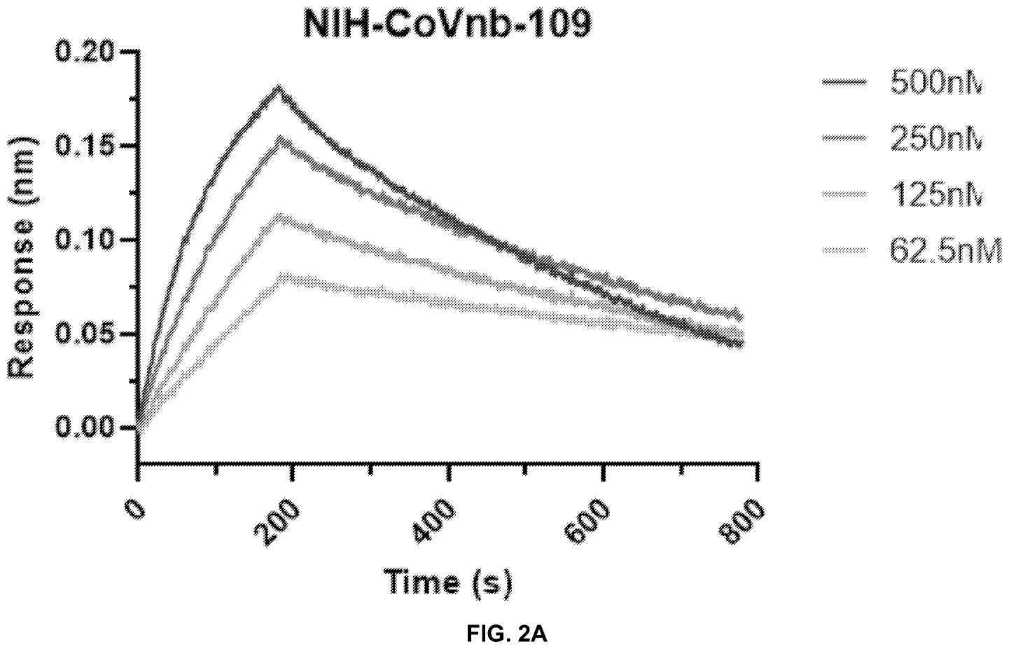

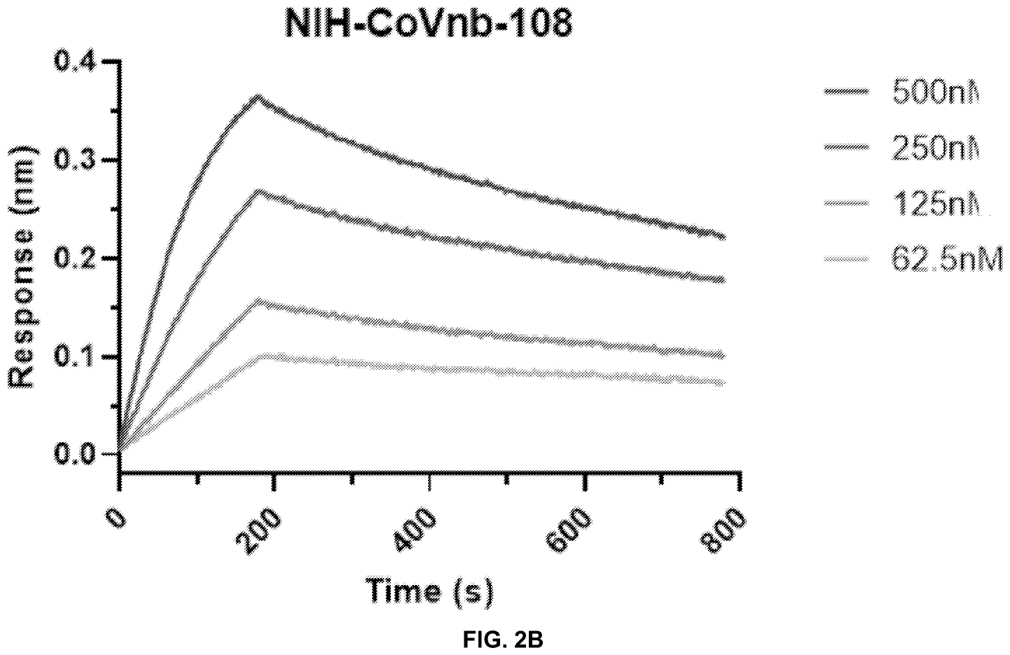

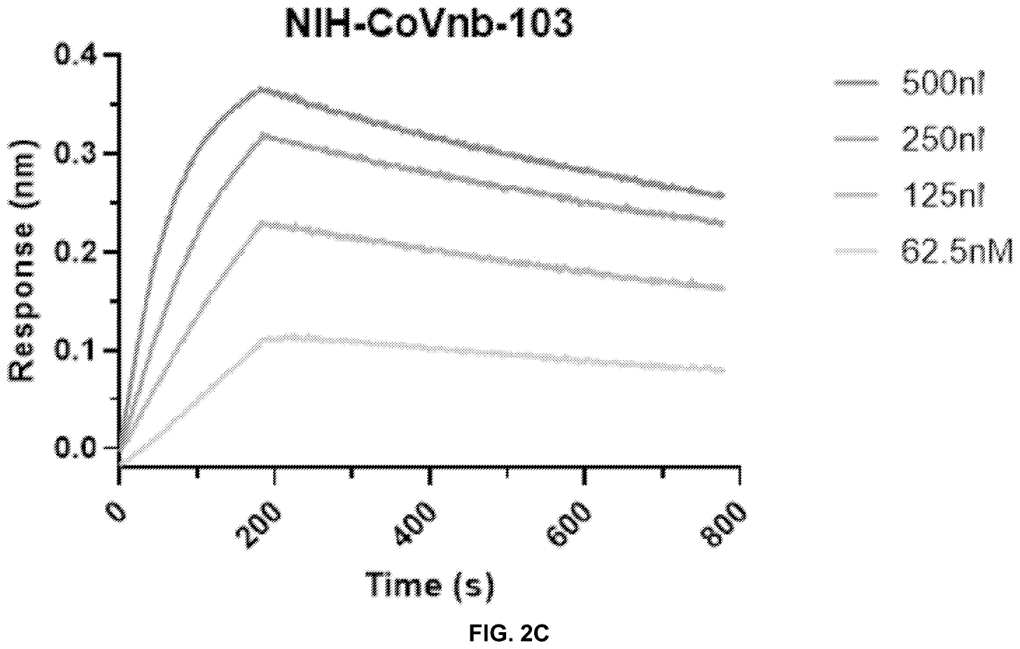

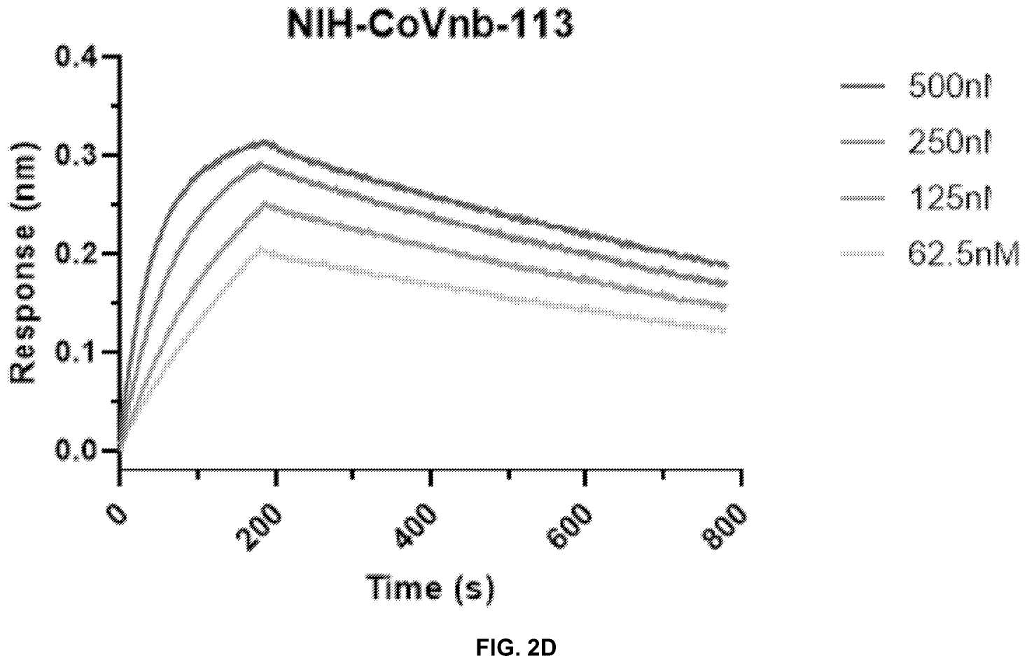

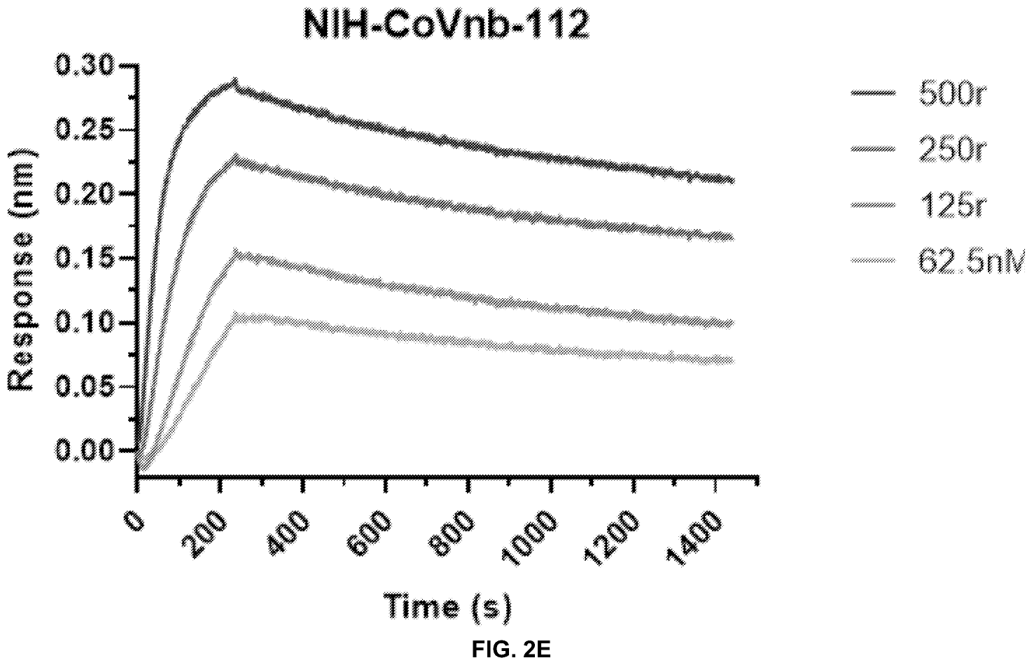

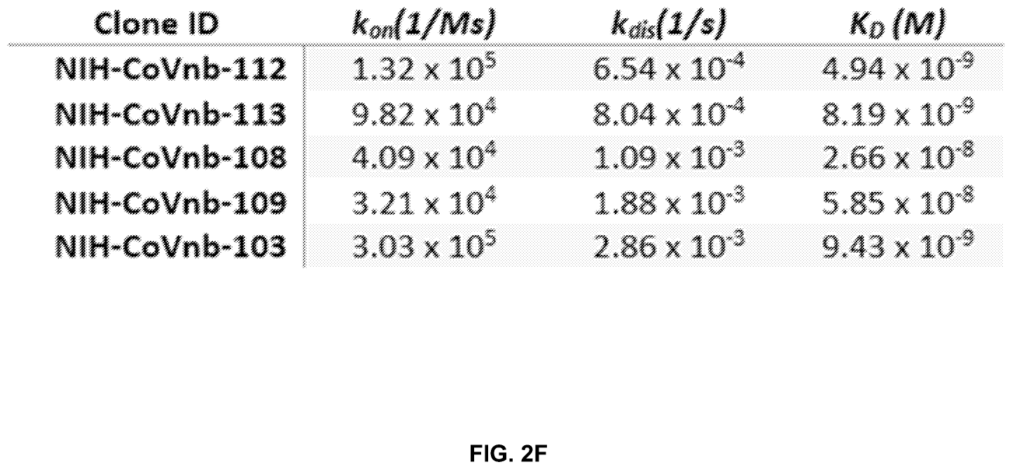

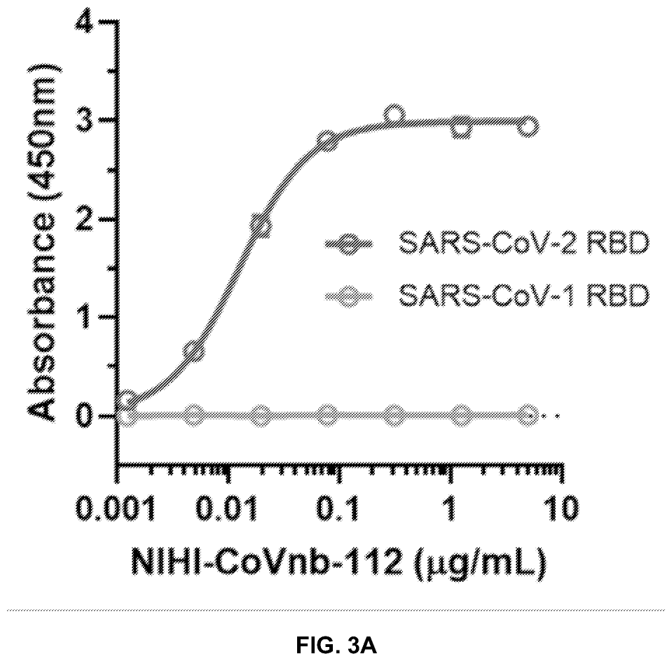

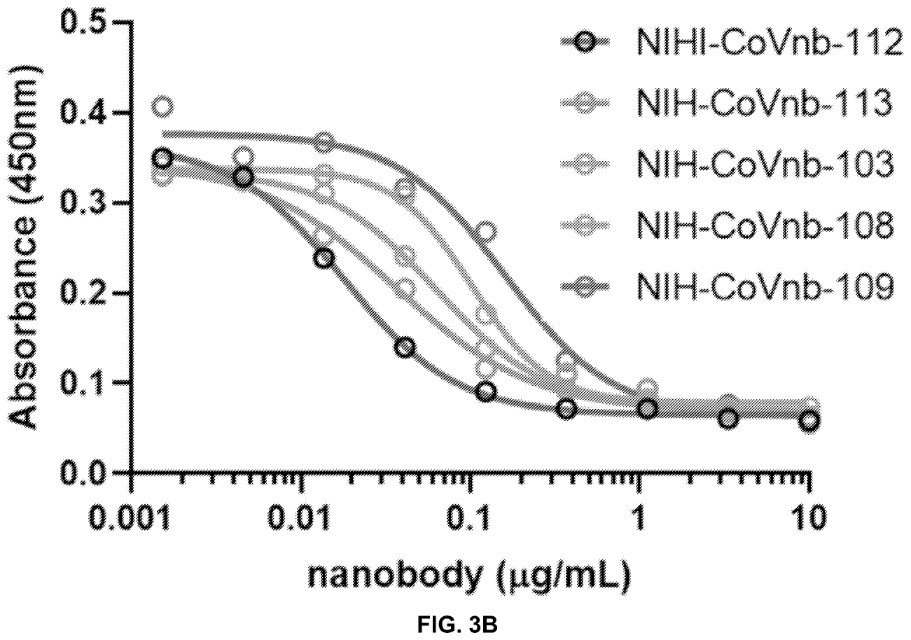

Anti-CoV-2 nanobody. Five anti-CoV-2 nanobodies were obtained from the camelid family (that includes llamas) which produce subclasses of IgGs possessing an unpaired heavy-chain variable domain, known as a nanobody. The nanobodies designed and described herein have been sequenced so they can be grown quickly and cheaply in bacteria for large-scale production, as well as be modified by recombinant molecular biology, e.g. to increase affinity or to orient them on the electrode surface, if needed. Nanobodies are generally hardier than antibodies; withstanding dehydration and larger temperature ranges, which could vary between sampling environments of the airborne detector described herein in conjunction with the biosensor electrode. The NIH-CoVnb-112 nanobody has an affinity of 5 nM and has a much higher selectivity for CoV-2 spike protein than CoV-1 in an ELISA format (FIG. 3A). Other nanobodies with lower affinity are also contemplated, depending upon the embodiment and sampling environment (FIG. 2F).

Airborne detection under realistic environmental conditions. Aerosol transmission is an important transmission pathway of CoV-2 on the basis of clinical observations in confined spaces. At present, there is a knowledge gap regarding the aerodynamic characteristics and transmission pathways of CoV-2 in aerosols because of challenges associated with their sampling in real-world settings and their quantification at variable particle sizes and concentrations. These real-world factors have direct impact on viral integrity and ability to be measured. Biosensor characterization includes a wide range of virus aerosol size and concentrations, including but not limited to different size distributions of virus aerosols corresponding to the different modes of airborne release via speaking, coughing, and sneezing. Aerosol samples are injected into an environmental chamber to mimic their fate and transport in a real-world indoor environment with pollutants. The biosensor development described herein encompasses a transformational change in the understanding of how environmental conditions alter airborne CoV-2 viral particles.

CoV-2 detectors. The CoV-2 immuno-based biosensor provides ultra-sensitivity for real-time pathogen detection. In some embodiments, the sensor detects CoV-2. Other embodiments include similar sensors developed with antibodies for other pathogens in a multi-electrode array. The airborne detector will enable continuous, instant feedback of a viral threat within the environment. It could flag an area for evacuation or increased disinfection later. It could also be coupled to a disinfectant aerosol spray for immediate resolution in order to keep crowds safe in real-time and limit disruption to on-going activities. Importantly, an airborne detector would alert that someone within a crowd is positive for COVID-19, possibly warranting individual testing within the group to identify and isolate on a much larger scale than currently is possible.

Anti-SARS-CoV-2 nanobody characterization. Five anti-SARS-CoV-2 nanobodies were raised to detect the repeat-binding domain (RBD) of the spike protein, as noted above. Using Biolayer Interferometry on a BioForte Octet Red96 system, association and dissociation rates were determined by immobilizing biotinylated-RBD onto streptavidin coated optical sensors to determine KON and KOFF of each nanobody (FIG. 2A-E). Curve fitting using a 1:1 interaction model allows for the affinity constant (KD) to be measured for each nanobody as detailed in (FIG. 2F). Various embodiments of the CoV-2 biosensor include these nanobodies, as well as several commercial monoclonal antibodies. One exemplary embodiment of the CoV-2 biosensor includes nanobody NIH-CoVnb-112, which has the highest affinity (˜5 nM).

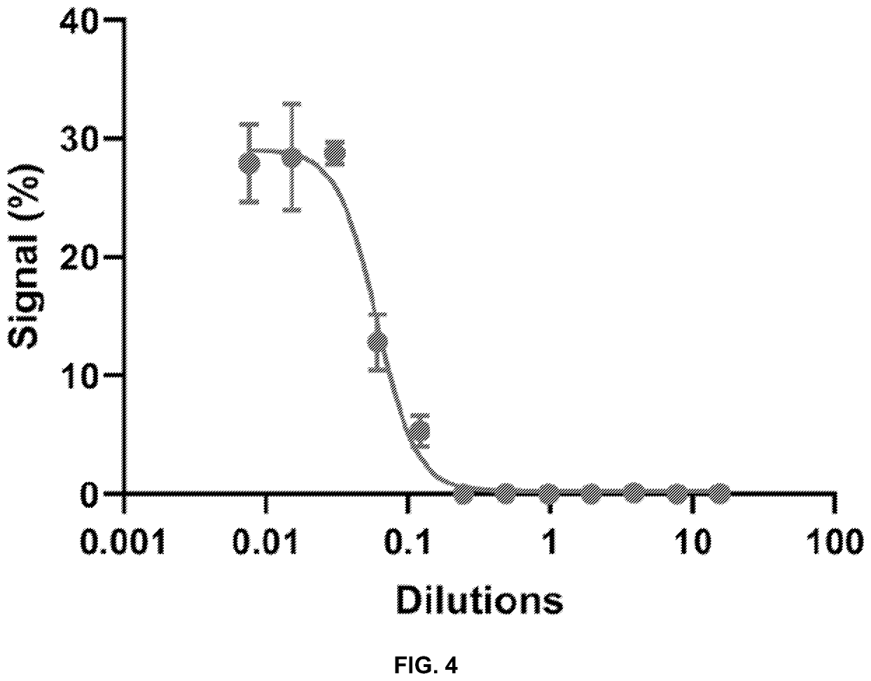

A direct ELISA was utilized to determine binding of NIH-CoVnb-112 to either SARS-CoV-2 RBD or SARS-CoV-1 RBD (FIG. 3A). The nanobody readily bound to the CoV-2 spike protein, although exhibited negligible binding to CoV-1 at any of the nanobody concentrations. A competitive binding assay demonstrates these nanobodies are capable of blocking CoV-2 from binding to ACE2 (FIG. 3B). NIH-CoVnb-112 produces the greatest inhibition of ACE2 binding with an EC50 of 0.02 μg/ml (1.11 nM). NIH-CoVnv-112 was also used to neutralize live SARS-CoV-2 virus from infecting Vero E6 cells (FIG. 4) in a FRNA50 assay, also demonstrating the virus' ability to bind intact viral particles, not just RBD protein.

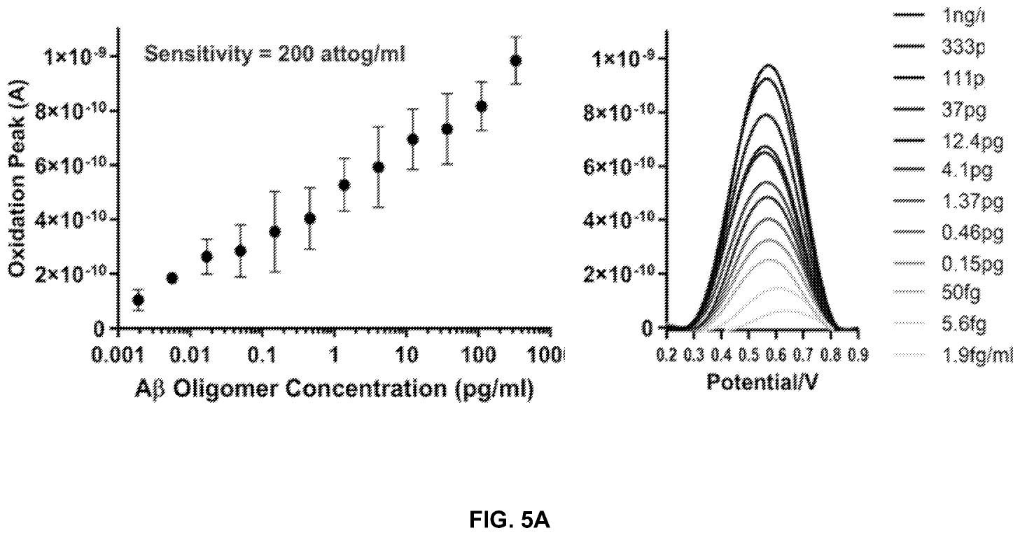

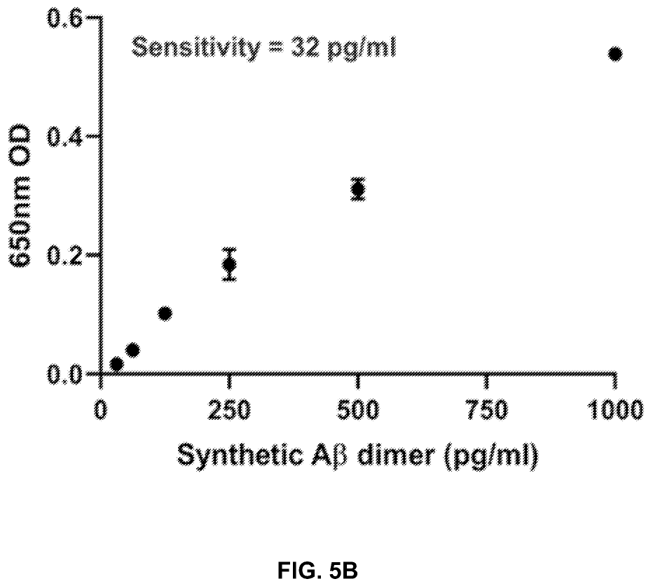

Anti-AB MIE biosensor. As mentioned herein above, an example of ultra-sensitivity of the MIE technology includes an AB MIE developed to detect oligomeric species using an aggregate-selective antibody attached to the carbon fiber electrode. The MIE detected AB dimers down to 200 attograms/ml (FIG. 5A). In contrast, commercial ELISAs to detect this Aβ oligomer target is sensitive to 32 pg/ml (FIG. 5B). Similar success was also observed in boosted sensitivity using the MIE for other target proteins, such as Aβ40 and tau.

CoV-2 biosensor. The MIE was coupled with NIH-CoV2nb-112 at 100 μg/ml, then incubated with a range of concentrations of CoV-2 spike protein RBD. The biosensor was sensitive to 2.0 fg/ml of CoV-2 RBD spike protein and saturated above 200 pg/ml (FIG. 6 (A-B)).

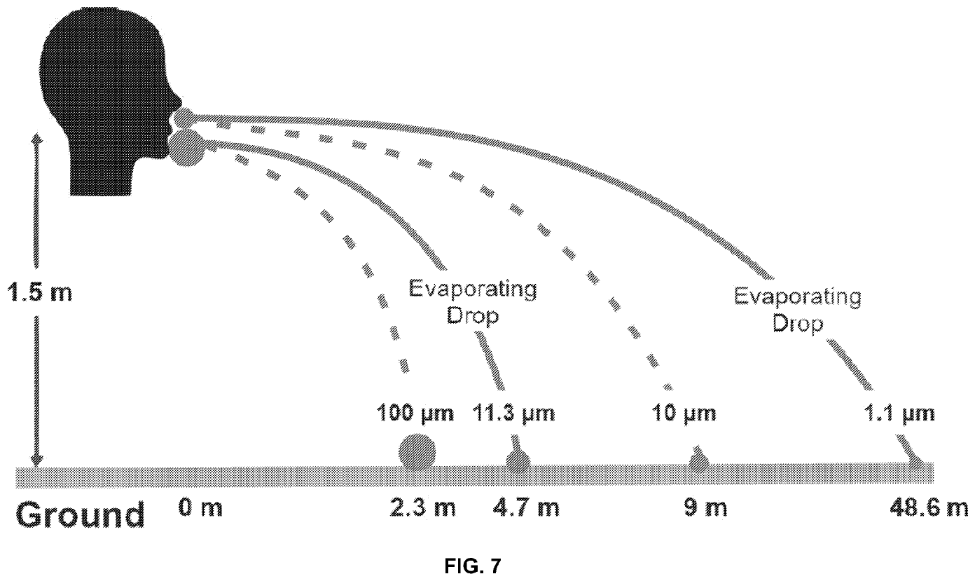

Aerosol dynamics and residence time. Airborne transmission encompasses both large particles and droplets (e.g. from speaking, coughing or sneezing) and smaller particles (e.g. due to evaporation). The transport, resultant lifetime, and fate of airborne droplets was numerically determined using the coupled governing equations of aerosol dynamics (such as droplet evaporation) and transport (diffusion, gravitational settling). FIG. 7 shows the horizontal distance traveled for droplets in the size range of 10 μm (green) and 100 μm (blue), respectively, at a relative humidity of 25%. Because of evaporation, the emitted droplets decrease in particle size thus increasing residence time, airborne lifetime, and horizontal distance traversed. For example, a 10 μm droplet will normally travel 10.9 m, but upon evaporation to 1.1 μm will travel 48.6 m. As such, the airborne detector disclosed herein has been designed with a particle-into-liquid sampler (PILS) that is able collect aerosol particles from 30 nm to 10 μm.

### General Methods

Production and inactivation of virus. SARS-CoV-2 (strains 2019-nCoV/USA-WA1/2020) and A/Puerto Rio/8/1934 (H1N1) virus are grown on Vero-E6 cells in a biosafety level 3 facility. Virus specificity tested include, in particular, other coronaviruses. Three days after inoculation, the supernatant is collected and pooled from several different tissue culture flasks. A first sample is taken for quantitative RT-PCR analysis to quantify the number of genomes in the supernatant. A second sample is taken to quantify the infectious titer by focus forming assay or plaque assay according to established protocols in the laboratory. To inactivate SARS-CoV-2 in the tissue culture medium, the fluid is incubated with a 1:1000 dilution of betapropiolactone (BPL) for 18 hours at 4° C. Inactivation of the virus is validated by focus forming or plaque assay. This tissue culture fluid containing SARS-CoV-2 particles can be used immediately for testing. In alternative embodiments, virus particle purification proceeds via ultracentrifugation on a sucrose gradient. In these embodiments, visualization by electron microscopy is performed to ensure minimal aggregation of viral particles that may be caused by ultracentrifugation, and further purified may be performed if necessary.

Target sensitivities of the CoV-2 biosensor. Depending upon the embodiment, the airborne detector biosensor's lower limit of detection for CoV-2 viral particles may vary based on the expected presence of CoV-2 particles in the sample. 75% of COVID-19-positive individuals have 105 CoV-2 viral particles in their sputum, whereas 50% and 5% have 10⁶ and 10⁸ particles, respectively.

Airborne CoV-2 detector. Exemplary embodiment of airborne viral load in a 10×10 meter room: Air in a well-ventilated room turns over 5-6 times per hour (every 10 minutes). An individual exhales 1 ml of EBC every 10 minutes, or 10⁵ CoV-2 viral particles from just breathing. Speaking or coughing could increase viral shedding by 6,000-fold. Virus from breathing is diluted into the entire room. A 10 m×10 m×2.7 m room has 270 L of air. In 10 minutes, an infected individual, within that 75% group, at rest could expel approximately 3.7×10² CoV-2 viral particles per L of air within the room. When the airborne detectors samples 20 L of air over a 5-minute period, 1.5×10⁴ viral particles are detected per 0.5 ml of test solution. In other embodiments, at least about a 50-fold lower sensitivity for the detector may be achieved, i.e., 6.0×10² viral particles/ml. Viral load would be much higher in a poorly ventilated room or if the individual was active in just about any way.

In some embodiments, the sensitivity target for an airborne virus detector is 2×10² CoV-2 viral particles/ml for the biosensor. Converting viral particle load to concentration, 2×10² viral particles/ml equates to 36 fg/ml of spike protein. The disclosed CoV-2 biosensor is sensitive to 2 fg/ml, making it already capable of the high sensitivity needed for these devices. Depending upon the embodiment, biosensor design includes suitable sensitivity, increased air flow rate, and/or sampling for longer periods of time to further increase signal.

Statistical methods are described and outlined herein below. When possible power calculations are used prior to an experiment to establish sample size, alternatively sample size calculations occur post-hoc. All experimental groups and run orders are randomized. Blinded studies and/or blinded data analyses are performed when feasible.

### Optimization of a CoV-2 Immuno-Based Biosensor

Immuno-based biosensor to detect CoV-2. The previously developed micro-immunoelectrode (MIE) biosensor as a platform for continuous measurement of the Aβ peptide with high sensitivity and specificity has been adapted to develop an immuno-based biosensor for CoV-2 by incorporating a CoV-2 specific antibody/nanobody attached to the electrode surface. Detection is achieved through the electroactivity of Tyrosine (Tyr) amino acids contained in the spike protein at positions 352, 365, 369, 380, 396, 421, 423, 449, 451, 453, 473, 489, 495, 505, and 508 bearing phenolic groups that can be oxidized at the surface of carbon-based electrodes. The oxidation pathway of Tyr can release an average of 3 electrons that are detected using square wave voltammetry (SWV), a technique in which the current at the working electrode is measured while the electrode potential is scanned through a range of 0V to 1.0V as a function of time. The advantage of SWV over an impedance measurement is that it provides a direct, instead of indirect, signal from the CoV-2 peptide itself. The voltammogram shows an increase/peak in measured current due to the oxidation of electroactive species, the location of the peak corresponds with the oxidation potential of specific species. Tyr oxidizes near a potential of 0.65V using carbon-based electrodes. While 0.65V can cause oxidation of a variety of molecules, including all nearby tyrosines, the antibody covalently attached to the electrode surface provides specificity for a particular target such as CoV-2. Peak oxidation currents are generated in less than 1 minute for rapid, continuous monitoring over a period of time. The height of the oxidation peak is proportional to the amount of protein at the electrode surface, allowing for relative concentration measurements to be obtained (FIG. 6 (A-B)). Increased specificity at the lower limit of detection lowers the false negative rate of testing. By monitoring the intrinsic electrochemical activities of the CoV-2 peptide, a direct, real-time (or near real-time), and reagent-less detection device is possible as shown in the devices, systems, and methods described herein.

Electrode preparation. In exemplary embodiments, biosensors are prepared by aspirating a single length of carbon fiber (5 μm diameter, GoodFellow Corp, England) into a glass capillary tube which is pulled into a fine tip using a pipette puller, the carbon fiber is attached to an insulated silver wire using conductive silver adhesive paste, sealed with heat shrink tubing, then cut to a length of 30-50 μm. To enhance oxidation of tyrosine and binding of the capture antibody, the microelectrodes are pretreated in PBS using a triangular waveform from 0 to 3V at 70 Hz for 20 s, followed by holding at −0.8V and 1.5V. Activation of carboxylic groups on the carbon fiber surface is achieved by application of 0.4M of EDC and 0.1M of NHSS solutions (Thermo Scientific, IL, USA) to form a semi-stable reactive amine NHS ester. The activated microelectrodes are placed in a solution of antibody and incubated at room temp for 10 min and then 4° C. overnight. Following antibody attachment, biosensors are incubated with 0.05% ethanolamine to deactivate reactive amine sites and then 0.1% albumin to block non-specific protein binding sites.

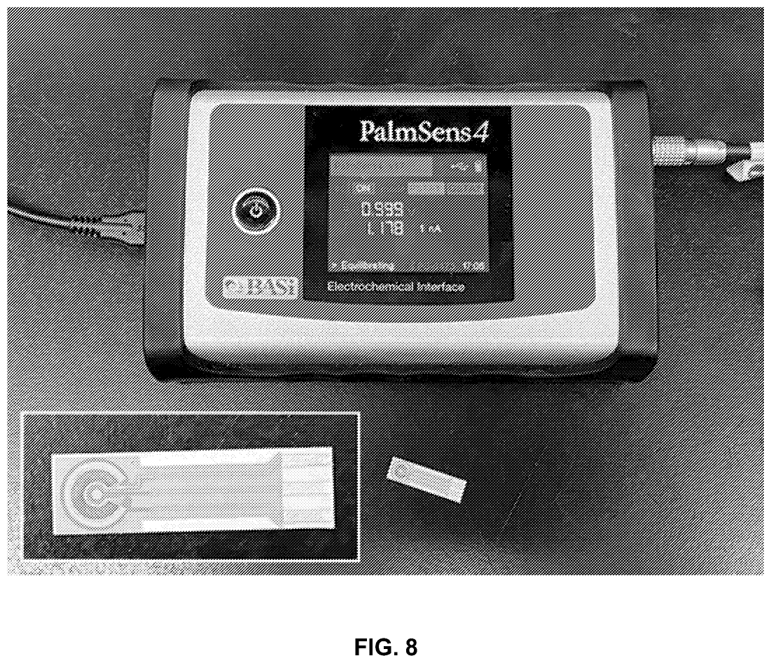

CoV-2 Biosensor. In exemplary embodiments, the biosensor has excellent sensitivity for CoV-2 RBD spike protein (2 fg/ml; FIG. 6 (A-B)) and is adaptable to detect intact viral particles, antibody type and concentration, as well as improved durability of the material for the desired environmental monitoring application. Depending upon the embodiment, biosensor design is optimized based on specificity, sensitivity, and longevity against inactivated CoV-2 viral particles. Controls include surface proteins of other viruses as well as other inactivated viral particles, such as influenza H1N1, H3N2, and H5N1 and other coronaviruses. Biosensors may be further designed to optimize for durability and increase production throughput, such as with screen printed carbon-based microelectrodes compatible with a commercially available product, e.g., PalmSens4 (BASi, Inc) potentiostat. The PalmSens4 is portable and can be run either connected to a computer via USB or on a smartphone via Bluetooth (FIG. 8).

Antibody/nanobody optimization. As disclosed herein, specificity is achieved by using anti-CoV-2 antibodies immobilized to the electrode surface (FIG. 1A), which facilitates detection of trace amounts of CoV-2 by concentrating the peptide at the electrode surface. The selectivity of the carbon fiber microelectrode demonstrates feasibility for use several environmental sampling applications, though the specific properties of the antibody/antigen binding kinetics influences the performance of the biosensor such that the biosensor can be adapted and/or optimized for the type of sensor desired. For example, an antibody that binds CoV-2 with high affinity will be useful for determining low levels in a sample. However, an antibody with weaker binding properties has the ability to release CoV-2 peptides after oxidation to have longer effective use time. Evaluation of CoV-2 specific nanobodies, several monoclonal antibodies, and control non-specific nanobodies aided in determining nanobodies which have the advantage of being more flexible in terms of orientation strategies and resistance to changing environmental conditions, compared to standard monoclonal antibodies.

Experimental Design. In some embodiments, CoV-2 specific nanobodies, two standard anti-CoV-2 monoclonal antibodies, and a control non-specific nanobody (raised against Aβ) with differing binding properties (FIG. 2F) are used to determine the most effective for longevity of repeated measurement in the environmental detection sensor. Two monoclonal antibodies having shown specificity for CoV-2 (Invitrogen, Inc) may be included, depending upon the embodiment. Each antibody is evaluated at high (100 μg/ml), moderate (20 μg/ml, and low (4 μg/ml) coating concentrations to determine optimal performance in solutions containing CoV-2 and CoV-1 spike protein, as well as samples containing CoV-2 and H1N1 viral particles to determine specificity of the signal (n=5 electrodes/condition). Biosensors are tested using spike proteins from 100 pg/ml to 0.1 fg/ml and 10 to 104 viral particles to determine the lower limit of detection with control to ensure specificity. Target specificity is at least 10×10² CoV-2 viral particles with 90% specificity (calculations and General Methods are described elsewhere herein).

Statistical Analysis. The lower limit of detection (LOD) and lower limit of quantification (LOQ) is calculated as 3 times the standard deviation of the measurements in blank sample, and similar for LOQ with a factor of 10. Intra-and inter-assay coefficients of variance (CVs) are calculated by the percent of variation between repeated runs of the same electrode within a sample, and across electrodes in the same sample concentrations. In some embodiments, only electrodes/antibody concentrations producing reliable CVs of less than 20% for both intra-and inter-assay variability are considered for further optimization. Specificity is determined by the lowest concentration at which measurements for CoV-2 are significantly greater (t-test p<0.05) than measurements with the same electrode in the same concentration of control. In exemplary embodiments, the antibody with the best specificity/sensitivity is chosen for the electrode design optimization and/or adaptation.

As disclosed herein, a high affinity antibody may provide the best sensitivity for trace detection in smaller spaces with time-point (e.g., non-continuous) monitoring, and a lower affinity antibody will provide longevity for repeated (e.g., continuous) monitoring in the environmental sensor. If the desired LOD or specificity at the LOD is not achieved using standard attachment protocol, a recombinant molecular biology can be used to modify the amino acids of the nanobody to specifically orient it when covalently-coupled to the biosensor surface.

Material Optimization. In some embodiments, biosensors are prepared using carbon fiber microelectrodes. In other embodiments, biosensors are prepared using a screen-printed carbon-based electrode to provide a more durable, low-cost electrode for use in the environmental monitoring equipment. Screen-printing of the microelectrodes also allows for flexibility in material used for the working electrode, for example incorporating carbon nanotubes or other nanomaterials to improve analytical performance of the sensor.

The NIH-CoV2nc-112 nanobody has been successfully attached and detected CoV-2 using a commercially available screen-printed graphite electrode (working diameter of 1 mm) connected to the portable PalmSens 4 potentiostat (FIG. 8). In exemplary embodiments, this electrode design is miniaturized to increase sensitivity for use with the sampling and/or monitoring equipment.

Experimental Design. Graphite working electrodes with Ag/AgCl reference and counter electrodes are screen-printed to a working diameter of 50 μm, 100 μm, and 300 μm. Screen printed electrodes are tested with the optimized antibody/nanobody attached and LOD and longevity are determined for each working electrode size (n=5 electrode/size). Similar to the above, results for LOD and LOQ are evaluated for each working electrode size to determine optimal sensitivity. To evaluate longevity, electrodes of each size are tested in continuous sampling of low or high concentrations of CoV-2 spike protein (SWV scan every 60 seconds) to determine reliability cut offs. When measurements become variable over 2 standard deviations from the starting point, the sensor is considered unreliable.

A smaller size working electrode provides higher sensitivity for low levels of CoV-2 protein, and is useful for smaller sampling space and/or non-continuous (e.g., sparse and/or randomized time-point sample) monitoring, while a larger size electrode provides extended (e.g., continued sampling) longevity useful for the environmental monitoring system. In embodiments where it is found that screen-printed graphite electrodes do not produce the desired level of sensitivity for CoV-2 detection as glass encased carbon fiber microelectrode, carbon nanotubes are incorporated into the electrode ink to enhance electron transfer and minimize fouling of the electrode. Alternative embodiments may include strengthening the carbon fiber design by encasing the glass seal in epoxy. In embodiments where it is found that longevity of the sensor does not exceed 8-12 hours (time limit determined to minimize repeated sensor changes), the time between sampling measurements is extended to determine if this extends sensor reliability. Criteria for electrode sensitivity, specificity, and reliability are described herein elsewhere.

### Environmental Detection of Airborne CoV-2

SARS-CoV-2 transmits via several modes, including aerosols and droplets, which remain suspended in air long enough to be inhaled. When aerosols or droplets containing respiratory fluid and microorganisms are expelled into unsaturated air, or air with relative humidity (RH) under 100%, they partially or fully evaporate to equilibrate with ambient conditions. As shown in FIG. 7, this process decreases the particle size and consequently increases its airborne lifetime. Evaporation also increases the concentration of free H+ions in an aerosol, which in turn, reduce the pH, while solutes such as salts and proteins remain intact. Interactions among salts, changing pH, temperature, and RH in a shrinking droplet are dynamic. Enveloped viruses, such as SARS CoV-2, that partition on the surface of aerosols may be subject to damage from increasing surface tension, shear stress, and conformational rearrangement driven by this dynamic interplay. Unfolding of peptides and subsequent denaturing of proteins can occur at the droplet's air-water interface. Therefore, proper consideration of environmental parameters such as RH and temperature is necessary in determining biosensor design, particularly because they affect the virus transmission dynamics (e.g. size, residence time, distance traveled) and their subsequent detection after they are expelled from infected individuals in the aerosol phase. The recovery and measurement of virus encapsulated in aerosol phase under different environmental conditions is significant for building a device that detects viral particles (e.g., CoV-2 viral particles) in a range of environments and conditions.

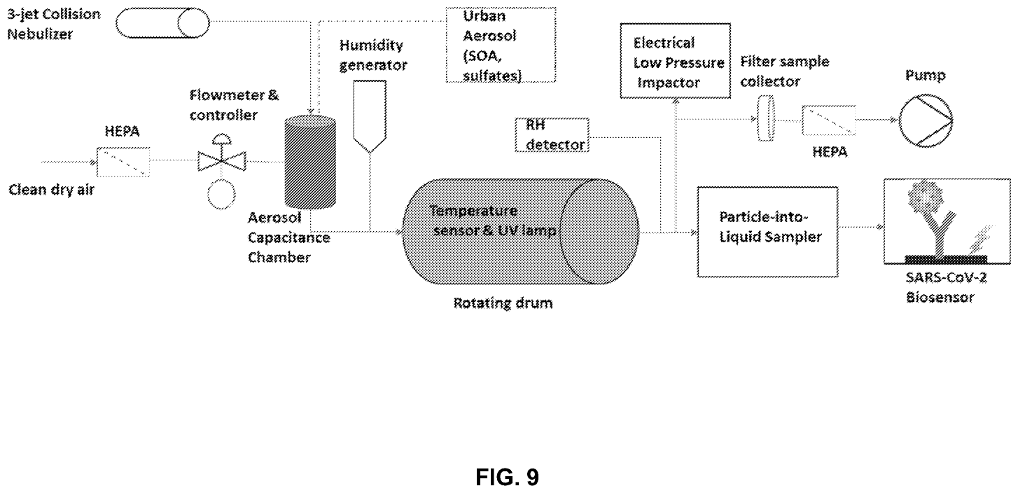

As described herein, aerosolization of inactivated SARS-CoV-2 suspensions into an exemplary system embodiment (FIG. 9) served to addressed the impact of sensor performance from: i) aging and environmental processing (RH, Temperature, and residence time) in terms of sensitivity, detection limits, and longevity (number of measurements); and ii) atmospheric particulate matter pollutants mixed with virus. To realistically simulate conditions in the natural atmospheric environment for studies of bioaerosol aging, the following combination was used: a) a reaction test chamber (rotating drum) with temperature and RH control ability to keep particles aloft for significant times (up to 24 hours); b) an aerosol generator to generate aerosol and inject them into the drum; and c) instruments for measuring particle size and number concentration, and sampling of particles into liquid media for offline injection into the biosensor.

Aerosol generation. A 3-jet Collison nebulizer (Biaera Technologies, LLC) operated at a pressure of 40 psi and a total flow rate of 12-L per minute (L/min) was used for aerosol generation. A droplet size distribution will be generated to mimic the three situations: exhaling (breathing), sneezing, and coughing of virus droplet release (see Table 1).

TABLE 1

| Modes of Airborne | Speaking | | |
|---|---|---|---|
| Release | (breathing) | Coughing | Sneezing |
| Number | 1.51 × 10⁵ | 2.37 × 10⁶ | 2.74 × 10⁸ |
| concentration | | | |
| (#/m³) | | | |
| Geometric Mean ± | 16.3 ± 3.6 | 13.4 ± 3.48 | 8.86 ± 2.21 |
| SD Diameter (μm) | | | |

For various embodiments described herein, aerosols contain vehicle (saline), inactivated CoV-2 viral particles, control viral particles (H1N1, H3N2, and H5N1), or combinations of CoV-2 and other viruses. The amount of viral particles is varied in solution between 10/ml and 106/ml to mimic a wide range of virus from an infected individual.

The nebulized virus aerosol stream first enters an aerosol capacitance chamber (ACC) that allows for mixing and initial evaporation of the droplets prior to their entering the rotating drum. HEPA filtered air is used as a carrier medium for the generated aerosol from the ACC to the drum. The aerosol laden air is then mixed with clean air that has been humidified using a Nafion drier (Perma Pure LLC, PD-50T-24), in order to control RH humidity at precise levels within the chamber. Controlling the ratio of clean humidified air to dry aerosol-laden air is used to produce various levels of sub-saturated RH conditions desired. The final conditioned airstream is introduced to the drum at a flow rate of 10 Lpm.

Rotating drum. Rotating drums have been used to maintain and study bioaerosol populations entrained in an air mass up to a few days. Briefly, the rotating drum design described herein is a 55.5-L aluminum cylinder with an interior diameter of 35.6 cm and a wall thickness of 0.48 cm that is rotated around a fixed center axle. The center axle is composed of clear acrylic and is divided into two concentric sections. The inner axle carries conditioned aerosol laden air into and out of the drum, and the outer axle serves as wire chase so that instrumentation can be fixed to the stationary mount on the axle. The targeted RH inside the drum is achieved by adjusting the flow rates of aerosol, dry air, and saturated air. A RH probe (HP-22A; Rotronic) monitors RH continuously in the drum. A temperature sensor probe located at the bottom of the right-side panel in the front of the enclosure is connected to the temperature control module; temperature can be adjusted and maintained at a range between 8° C. and 45° C. The middle of the center axle holds a UV light socket for a UV-C lamp emitting at 254 nm. The lamp is plugged inside the aerosol chamber at the center of rotation and can emit radiation at a 360° angle.

Particle-into-liquid sampler. Solvent-soluble aerosols are collected using a particle-into-liquid sampler (PILS) prior to virus detection by the biosensor. There are several means to collect aerosol particles into liquid solution (e.g. impactor, condenser, etc.), and the choice of collection technology would determine the size of aerosols being collected. A typical breath produces aerosols within the 0.03-10 μm range, aerosols change in size based on environmental conditions before being collected into the CoV-2 detector. Thus, it is important to use the appropriate technology to capture as wide a range of aerosols as possible.

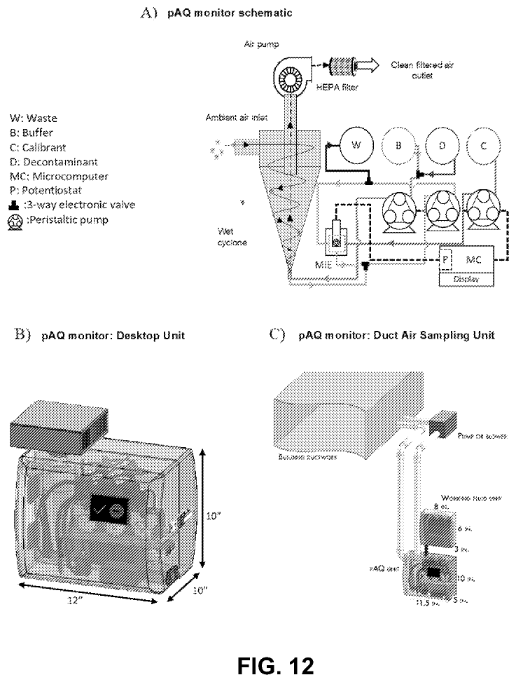

A custom PILS was designed, which uses the “wet cyclone” technology for particle collection. With this technology, sampling of ambient aerosols in the aerodynamic size range of 0.03-100 μm is ensured. The wet cyclone PILS operates at a wide range of flow rates (50-1000 liters per minute) and user-defined sample collection times (that can be as low as 5 minutes). However, operating the wet cyclone PILS at lower flow rates could influence overall device performance, since the particle collection efficiency of the wet cyclone is depended on the flow rate and inlet air velocity. The wet cyclone (FIG. 12A) is connected to a high-flow vacuum pump to sample air at ˜1,000 (±10%) liters per minute (lpm). Prior to air sampling, the cyclone is filled with a predefined volume (˜15 ml) of phosphate-buffered saline (PBS) solution. The pressure drop rapidly draws in ambient air through a tangential inlet creating a vortex, which produces a rotating film of PBS liquid on the inner wall of the cyclone. Aerosols entering the wet cyclone impact the inner wetted walls and are collected in the liquid media. Aerosols not captured by the wet cyclone exit from the top and are captured by a HEPA filter. Air is sampled for 5 min, after which the concentrated particles collected inside the PILS are then transferred to the biosensor using an automated liquid delivery system for the final SARS-CoV-2 detection. The PILS has an inbuilt automated HOCl decontamination feature that decontaminates the entire device prior to its disassembly or shutdown. This self-decontamination feature ensures that the users are not exposed the user to any of the potentially hazardous bioaerosols collected inside the PILS.

Testing protocol. Virus or biosphere laden aerosol are continuously introduced into the chamber for 30 min to reach varying degrees of aerosol particle number size concentrations (between 102 and 105 per Liter). The aerosol concentration are monitored using the aerosol number size distribution instrument—Electrical Low Pressure Impactor (ELPI; Dekati Inc.). Once the threshold concentration is reached, the filter and PILS samplers are used to sample aerosol from the drum. The measurements are repeated approximately every half hour, with no air being withdrawn from the drum between sampling periods. Once a sample measurement is finished, and before a new sample is started, the chamber is evacuated at 40 L/min with dry HEPA-filtered air until no particles are detectable using the ELPI.

Regarding impact the sensor's performance from aging and environmental processing (RH, Temperature, and residence time) in terms of sensitivity, detection limits, and longevity (number of measurements). Relative humidity is varied across a wide range from 20% to ˜100% in steps of 10% with 30 minutes per step. Air is sampled from in increments of 1, 5 and 10 minutes at each step to test biosensor sensitivity. Past studies have observed a distinct U-shaped pattern regarding the relationship between RH and the viability of virus droplets. Viruses seem to survive best at both low (<40%) and extremely high (>90%) RH, while they experience significant decay at intermediate RH.

Temperature is varied ranging from 10° C. to 50° to mimic real-world conditions. When the first outbreak of COVID-19 was reported, the temperature in Wuhan, China was around 17° C. in the morning and 8° C. at night. Tropical countries where the disease persisted had over 40° C. temperature. A recent molecular dynamics simulation study has shown that temperature is a very significant variable because spike glycoproteins respond differently in high and low temperature conditions.

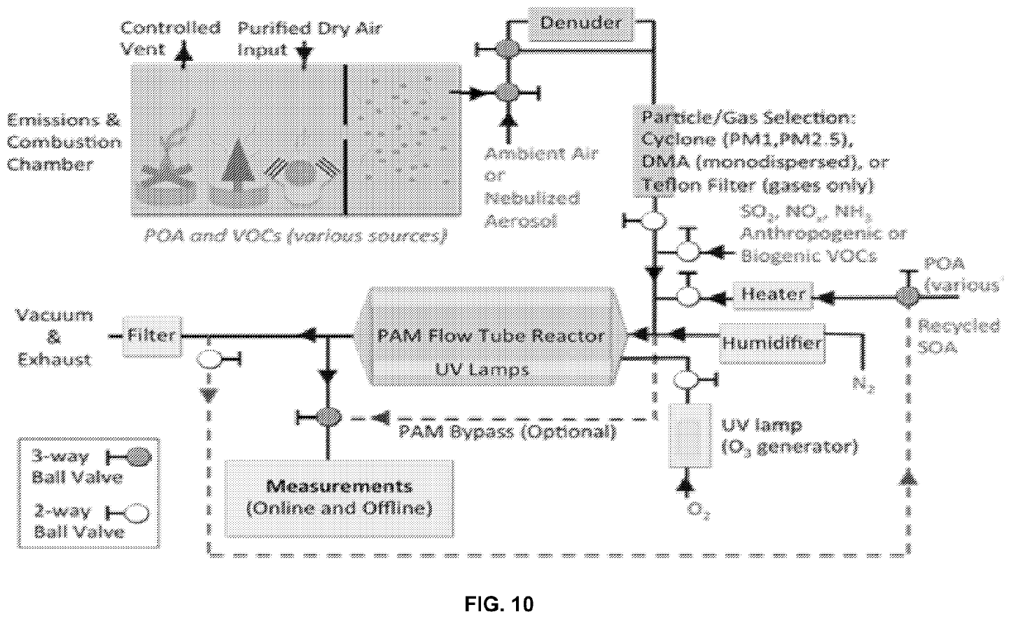

Regarding effect on the sensor performance from mixing of the virus aerosol with atmospheric particulate matter pollutants. Airborne viruses and fine particulate matter air pollutants interact to influence viral stability and infectivity. The pollutant particles may also act as carriers, which could have complex adsorption and toxicity effects on the pathogen. Laboratory-generated particulate matter pollutants, such as 1) ammonium sulfate (emitted from vehicles and power plants), 2) secondary organic aerosol (emitted from volatile organic compounds (VOCs) in urban indoor environments), and 3) primary organic aerosol (POA; emitted from wildfire), are introduced into the ACC (shown in perforated lines in FIG. 10) in parallel with nebulized virus aerosol stream.

An exemplary system embodiment setup for generating atmospherically relevant aerosol is summarized schematically in FIG. 10. A novel emission/combustion chamber has been designed and constructed to allow for combustion or thermally-driven emissions of organic gases and particles from a desired source input (e.g. biogenic, urban, wildfire). Separate sub-chambers are available within the emissions/combustion chamber to allow for combinations of sources to mimic a complex environment at different ratios. The resulting sample stream is sent through either a cyclone, a Differential Mobility Analyzer (DMA) to provide monodisperse aerosol, or a Teflon filter to remove particles to further control the environment. The resulting primary sample (gas or particle) has the option of mixing with a range of other pure components at controlled concentrations (e.g., SO₂, NOₓ, NH₃, anthropogenic or biogenic VOCs) as well. The benefit of this setup is the ability to create any combination of gas and particle input from any source type or mixture of source types that can contribute to atmospheric emissions, with the addition of being able to work directly with ambient air if desired.

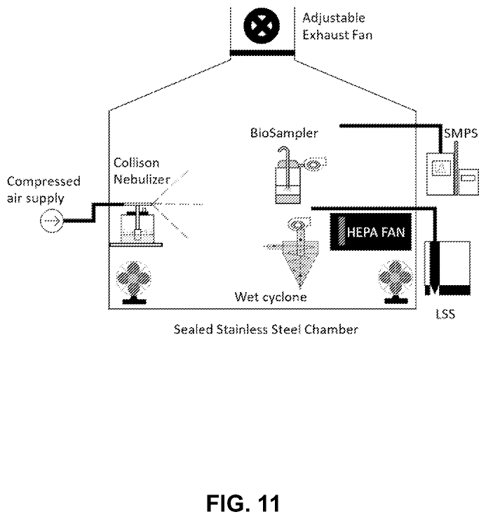

An exemplary system embodiment setup for generating laboratory inactivated virus aerosol is summarized schematically in FIG. 11. A novel emission/combustion chamber has been designed and constructed to allow for aerosolization and mixing of different test aerosols. The benefit of this setup is the ability to sample the air using multiple PILS for direct in parallel intercomparison studies.

CoV-2 biosensor for airborne detection. In an exemplary embodiment, the CoV-2 biosensor includes an optimized electrode as described above. Also as noted above, 75% of individuals with COVID-19 will release 10⁵ viral particles over 10 minutes of normal breathing. For the airborne detector as described herein sampling 20 L of air over a 5-minute period, 1.5×10⁴ viral particles are detectable per 0.5 ml of test solution. In another exemplary embodiment, a 50-fold lower sensitivity is 6.0×10² viral particles/ml, or target sensitivity is designed for the airborne detector. The conditions necessary for the biosensor to detect CoV-2 or negative control viruses namely focus on length of collection time (5, 10, or 20 minutes of air). Increasing the length of time improves sensitivity, yet may reduce measurement frequency. At a given collection rate, repeated measurements determine longevity of the biosensor. An exemplary embodiment includes an electrode that takes continual measurements at a target sensitivity for a minimum of 12 hours; preferably 24 hours. In some embodiments, the target specificity to exclude other viruses is greater than about 85%, greater than about 90%, greater than about 95%, or greater than about 99%.

A mass balance equation is used to correct for loss of aerosols via gravitational settling and dilution during aging, assuming first-order decay for both processes inside the drum. Charge generation by the rotation of the drum has been observed to cause the loss of particles during past experiments. To circumvent this issue, a small Polonium source (Amstat Industries Inc., 2U500) is inserted inside the drum on the axis to provide additional ions for neutralization, and grounded copper strips are added inside and outside the drum chamber along the supporting wheel tracks to conduct any charge generated by the turning drum to ground. In the event of the drum malfunctioning, an environmental chamber (5518; Electro-Tech Systems) operated at room temperature (22° C.) and the same RH values listed above for aerosol experiments is used. The PILS collector is impacted by noise and drift, and is initially to corrected for if needed.

The CoV-2 biosensor in the airborne detector must have both sensitivity and longevity for repeated measurements. If needed, sensitivity can be modulated by using an antibody with different affinity, altering the concentration of antibody on the electrode surface, or modulating the sample time of air. Longevity is often a factor of antibody affinity; decreasing the affinity, either by using a different antibody or targeted mutations of the nanobody to alter its binding properties.

### Method and Device to Interface an Ambient Aerosol Sampler With an Electrochemical Biosensor for the Detection of Airborne Pathogens

As disclosed herein, a novel method has been developed for the interfacing of any aerosol-to-liquid sampler with an electrochemical biosensor, referred to as a pathogen Air Quality (pAQ) monitor. Deployment of this method embodies a rapid (e.g., a real-time or near-real-time) continuous airborne pathogen (e.g., viruses including coronaviruses and/or other respiratory viruses, bacteria, fungi, mold, and/or parasites) detector for monitoring of indoor spaces, including large indoor spaces. In some embodiments, the airborne detection system detects SARS-CoV-2. In other embodiments, the airborne detection system detects coronavirus, including influenza. In yet other embodiments, the airborne detection system detects at least virus, bacteria, fungi, mold, and/or parasite.

In an exemplary embodiment, a proof-of-concept device (FIG. 11), referred to as a pathogen Air Quality (pAQ) monitor, that comprises a wet-wall cyclone coupled to an MIE detection unit that houses an automated liquid handling unit and MIE biosensor assembly (FIG. 11). The inbuilt decontamination feature of the pAQ monitor enables contact-free system decontamination and ensures safe handling of the device.

In at least one exemplary embodiment, the device is a desktop instrument (FIG. 11B). A touchscreen interface on the front panel includes data on the electrode status (time to replacement, presence of virus, etc.), fluid levels, pump activity, and ambient variables such as temperature and humidity. Some embodiments further include miniaturization to reduce power and fluid consumption and facilitate installation in areas where space may be limited.

In at least one exemplary embodiment, the device is an air duct sampling unit (FIG. 11. B). A touchscreen interface on the front panel includes data on the electrode status (time to replacement, presence of virus, etc.), fluid levels, pump activity, and duct air flow variables such as temperature and humidity. Some embodiments further include miniaturization to reduce power and fluid consumption and facilitate installation in areas where space may be limited.

Internally, the system of present disclosure consists of a fluid network driven by pumps, with internal reservoirs for replenishment fluid and waste fluid (FIG. 11). A specially-prepared electrode resides in the analysis vial and is electrically connected to a custom-programmed OEM electrometer. Pumps are controlled by an internal computer which also updates and responds to the user interface.

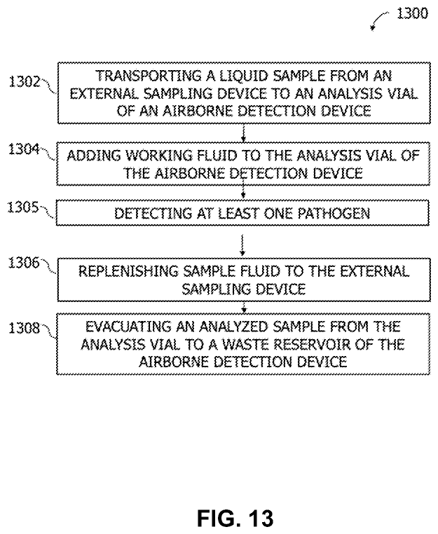

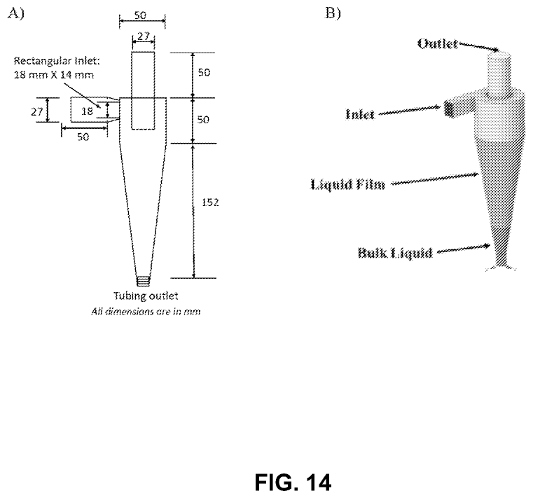

In some embodiments, a method (1300, FIG. 13B) comprises transporting 1302 a liquid sample from an external sampling device to an analysis vial of the airborne detection device. The liquid sample is pumped from the external aerosol-to-liquid sampler to the analysis vial (FIG. 14).

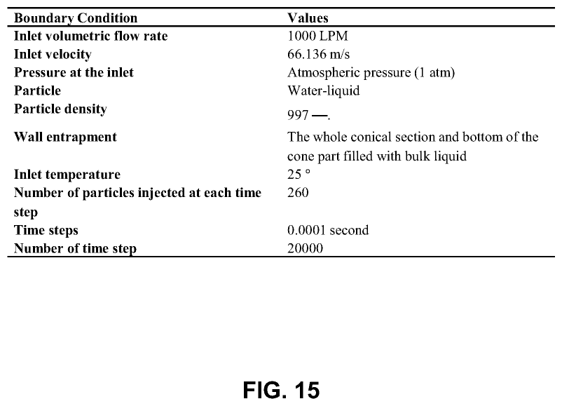

Method 1300 further includes adding 1304 working fluid to the analysis vial of the airborne detection device. Additional working fluid is added (if necessary) to the analysis vial (FIG. 15). 2. If viruses are present, method 1300 still further includes detecting 1305 at least one pathogen. A reaction occurs, and the electrometer registers a signal from the electrode. The user interface updates and if necessary, displays an alarm which also appears on the network output.

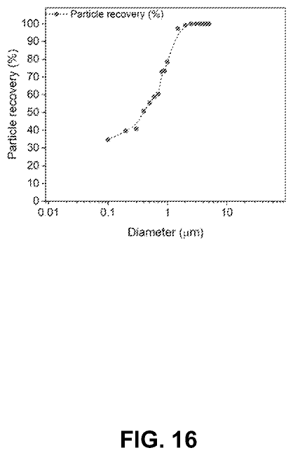

Method 1300 also includes replenishing 1306 sample fluid to the external sampling device. Sample fluid is replenished to the sampler (FIG. 16).

Method 1300 additionally includes evacuating 1308 an analyzed sample from the analysis vial to a waste reservoir of the airborne detection device. After the sample is analyzed, it is evacuated to the waste reservoir. If no virus was detected, sufficient working fluid is added to the analysis vial to maintain the electrode.

In this embodiment, the electrode takes approximately five minutes to analyze a sample. While it is analyzing, the external sampler gathers the next sample. Depending on the embodiment, the electrode may take less than about 10 minutes, less than about 5 minutes, less than about 4 minutes, less than about 3 minutes, less than about 2 minutes, or less than about 1 minute to analyze a sample.

### Numerical Simulations of the PILS Wet Cyclone to Calculate its Theoretical Particle Collection Efficiency

In a high-flow rate cyclone, the particle separation depends on the swirling flow pattern of the incoming particles. The swirling airflow forces the particles to impact the cyclone wall, which drives particle separation. The flow regime inside a cyclone is highly turbulent and anisotropic. The carrier phase is the gas (ambient air), and the dispersed phase is the particle (water-liquid). We used Ansys Fluent 2021 R1 to perform the numerical simulations. Reynold stress model was applied for modeling the flow field in the cyclone body.

The solution algorithm used for solving the pressure-linked equations is “SIMPLE”, and spatial discretization applied is as follows: “PRESTO!” discretization for pressure, “QUICK” method for momentum equation, second order upwind for turbulent equations, and first order for Reynold stress terms. ICEM-CFD was used to create a structured hexahedral mesh of the cyclone design.

FIG. 14A shows the dimension and FIG. 14B shows the computational domain of the simulated wet cyclone. The volumetric flow rate at the cyclone inlet was set as 1000 lpm (equivalent to 66.138 m/s inlet velocity). The boundary conditions assumed for this simulation are summarized in FIG. 15. As shown in FIG. 14B, we assumed a thin liquid film forms along the walls of the cyclone. The bottom portion of the wet cyclone (50 mm) was assumed to be filled with liquid (based on experimental observation). It is assumed that particles coming in contact with the cyclone wall (conical section) or liquid surface at the bottom of the cyclone are trapped. To make sure that there is an equal mass between the inlet and outlet, the average volumetric mass between the inlet and outlet was monitored during the simulations. FIG. 16 shows the particle trapped inside the wet cyclone for sizes ranging from 0.1 to 5 μm The numerical model results show that the wet cyclone has >95% collection efficiency for particles >1 μm and a cutoff diameter (where the collection efficiency is 50%) of 0.4 μm.

### PILS Wet Cyclone Virus Aerosol Sampling Performance in Laboratory: Chamber Aerosolization Experiments

The wet cyclone virus sampling performance was compared with two commercially available PILS: a BioSampler® (SKC Inc., USA) and a Liquid Spot Sampler™ (LSS; Aerosol Devices, USA). The PILS intercomparison experiments were performed by aerosolizing inactivated Washington strain (WA-1) of the SARS-CoV-2 virus inside a well-mixed 21 m³ sealed stainless steel test chamber (FIG. 11). The wet cyclone, LSS, and BioSampler® were set up to sample the air inside the chamber simultaneously for 10 minutes. Note, while the high-flow wet cyclone requires <5 min of sampling for virus detection, we performed the chamber experiments for 10 min to ensure sufficient virus concentration was collected in the BioSampler® and LSS for RT-qPCR analysis. After sampling, the virus collected in each device was quantified using RT-qPCR. To study the influence of initial virus concentration on sampling performance, we performed the chamber experiments at three virus loading conditions: <500 copies/m³ (“low”), 500-10,000 copies/m³ (“medium”), and >10,000 copies/m³ (“high”). All experiments were performed either in duplicate or triplicate runs.

FIG. 11 shows the basic layout of the 21 m³ chamber experiment setup. 100 μl aliquots of the stock virus were thawed and diluted in PBS to desired concentrations. The Collison nebulizer (CH Technologies, USA) filled with inactivated WA-1 (diluted in PBS) was placed in one corner of the chamber. The nebulizer outlet was at a height of ˜3 feet from ground level. Two fans were placed on opposite corners of the chamber to achieve uniform mixing inside the chamber (fan height=1-1.5 feet). The wet cyclone and the BioSampler® were placed inside the chamber, approximately at the center, but sufficiently far apart to not interfere with either of their sampling collection (chamber floor area: 7.13 m² or 11′×7″). The stainless-steel test chamber has multiple welded stainless-steel ports (¼″ diameter) that can be connected to external air sampling devices for chamber air sampling. We connected one of these sampling ports to the LSS and another to the Scanning Mobility Particle Sizer (SMPS; TSI Inc. USA). We ensured that all the sampler inlets were at similar heights so that all the instruments sampled air at the same height (4 to 4.5 feet from the floor).

### Experimental Procedure

(1) Chamber cleaning: The exhaust fan shutter is completely opened to facilitate maximum ventilation. The vertical HEPA fan filter (80 CFM, 99.97% particle removal) is switched on to filter out all particles suspended inside the chamber. The chamber is cleaned for 60 minutes before the start of any experiment. The size distribution and particle number concentration (PNC) inside the chamber are continuously monitored using the SMPS. (2) The Collison nebulizer filled with the stock virus was then connected to a 20-psi compressed particle-free air supply. The nebulizer was left running for 5 minutes to obtain stable aerosol generation. (3) After 5 minutes, all three samplers were switched on, and air sampling was performed for 10 minutes. (4) The three samplers and the Collison nebulizer were turned off after 10 minutes of sample collection. Step 1 was repeated, and the PNC was monitored using the SMPS. (5) When PNC reached its original background concentration, the door to the chamber was opened, and the samples were collected and stored for RT-qPCR analysis inside a refrigerator at 4° C. All RT-qPCR PCR analyses were completed within 18 h of sample collection.

### Wet Cyclone PILS Virus Recovery

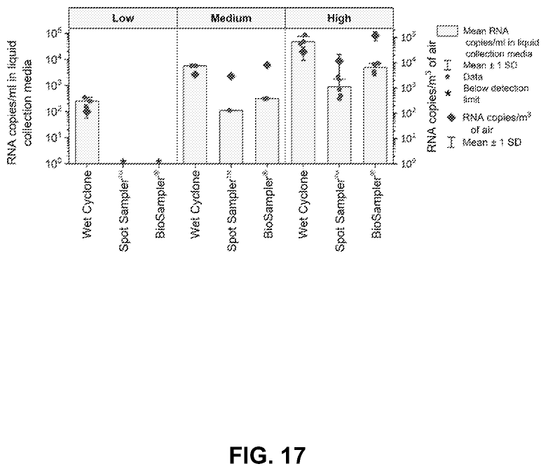

FIG. 17 compares the virus recovery of the wet cyclone with the BioSampler® and LSS inside a sealed chamber at low, medium, and high aerosolized WA-1 concentrations. After 10 min of sampling, the viral RNA concentration measured by the wet cyclone (i.e., RNA copies/ml of collection media) was, on average, 13 and 66 times higher than the concentration measured in the BioSampler® and LSS, respectively. Interestingly, under low concentration conditions, the WA-1 RNA was recovered only in the wet cyclone, whereas the samples collected inside the BioSampler® and LSS were too low to be quantified by RT-qPCR. The high RNA recovery by the wet cyclone can be attributed to its extremely high flow rate, which allows it to sample a larger volume of air (˜10 m³) during 10 min sample collection compared to the BioSampler® (˜0.125 m³) and LSS (˜0.015 m³). This characteristic makes the wet cyclone ideal for use in high-time resolution continuous monitoring applications in real-world environments such as hospitals and patient isolation rooms, where the airborne virus concentrations could vary from 2-94,000 copies/m³.

### Virus Aerosol Sampling Performance in Infected Households

The wet cyclone assembly (the cyclone, vacuum pump, and PBS solution) was shipped to the apartments of two volunteers who were confirmed SARS-CoV-2 positive. The volunteers collected 5 min air samples (n=3 to 4) from inside their bedrooms/apartments and then stored them in 15 ml centrifuge tubes on ice. The liquid samples were then transported to a laboratory and analyzed using RT-qPCR to detect the presence of SARS-CoV-2.

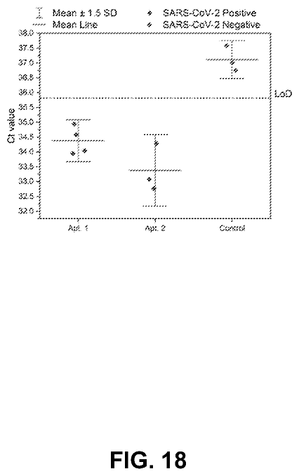

All seven air samples collected using the wet cyclone in the two apartments occupied by SARS-CoV-2 patients tested positive based on RT-qPCR (FIG. 18). The RT-qPCR results of bedroom samples were compared with air samples collected from a virus-free control room. The Ct values of the air samples from the infected households ranged from 32.7-34.9. In contrast, no virus was detected in the control air samples (concentration<instrument LoD). The significantly lower Ct values observed in the apartment air samples compared to the control air indicate the possible presence of SARS-CoV-2 in the apartment air.

### pAQ Monitor LoD

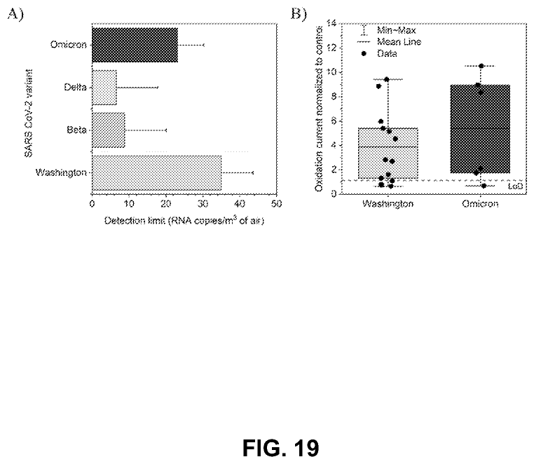

The LoD of the pAQ monitor is calculated using the SARS-CoV-2 strain specific biosensor LoD data explained earlier and normalizing it with volume of air sampled per ml of the liquid collection media. FIG. 19A shows the SARS-CoV-2 variant-specific LoD of the pAQ monitor for 5 min air sampling. The pAQ monitor LoD was 35, 7, 9, and 23 RNA copies/m³ of air for the WA-1, delta, beta, and BA-1 strains, respectively. Note that these values only apply for virus aerosols >1 μm (˜100% collection efficiency). The LoD for the virus in the submicron-sized aerosols will vary based on the wet cyclone particle size-dependent recovery fraction (FIG. 16).

### pAQ Monitor Virus Detection Sensitivity

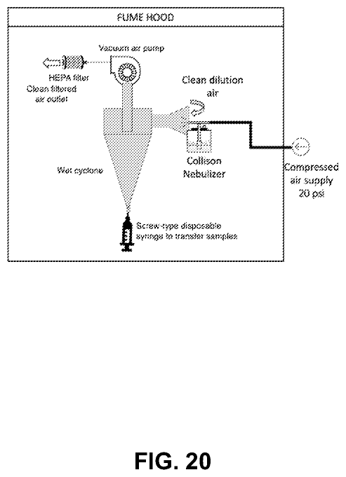

Different concentrations of test SARS-CoV-2 were prepared by diluting inactivated SARS-CoV-2 stock in PBS solution. The test solution was aerosolized using a Collison Nebulizer inside a fume hood (FIG. 20). The Collison aerosol generation port was placed directly in the center of the wet cyclone inlet, using custom built wide mouth glass conical adaptor (˜4″ diameter opening). Here, we assume the strong suction at the inlet of the wet cyclone will ensure that all the aerosols generated by the Collison nebulizer enter the cyclone. Along with the virus aerosols, ˜993 L of clean lab air also enters the cyclone. The wet cyclone was filled with 15 ml PBS solution, and 5 min aerosolized virus sampling was performed. After 5 min, the sample was taken out manually using a disposable syringe screwed onto the base of the wet cyclone. The samples collected were manually divided into two portions, one was analyzed using the biosensor, and the other was analyzed by RT-qPCR (within 18 h of sample collection). After retrieving the samples, the wet cyclone was decontaminated using 70% ethanol. The wet cyclone was washed, rinsed with milli-q water, and air-dried before the next round of sample collection. FIG. 19B shows the pAQ monitor performance when sampling laboratory aerosolized inactivated WA-1 and BA-1. pAQ monitor showed a sensitivity of 77% for WA-1 and 83.3% for BA-1. The virus sensitivity of the pAQ is comparable to the sensitivity of other recently developed rapid biosensors (<10 min detection time) used for detecting viruses in saliva, nasal swabs and exhaled breath condensate samples.

### pAQ Monitor Design for Duct Air Sampling

In at least one exemplary embodiment, the device is a duct air sampling instrument (FIG. 12C). In this configuration, the wet cyclone inlet is connected to the air duct of a building ventilation system. The air from the building duct will be sampled using a ring blower or vacuum pump connected in line with the pAQ monitor. In this configuration, the pAQ air sampler is split into three independent modules: (1) pAQ Monitor Unit, (2) Working Fluid Unit, and (3) Air Pump.

pAQ Monitor Unit consists of the wet cyclone sampler, a touchscreen interface on the front panel includes data on the electrode status (time to replacement, presence of virus, etc.), fluid levels, pump activity, and ambient variables such as temperature and humidity. Some embodiments further include miniaturization to reduce power and fluid consumption and facilitate installation in areas where space may be limited. The unit will also house a custom automated biosensor loading device. This automated biosensor loading device will hold 16-48 (or higher) pre-calibrated biosensors which will be periodically replaced, as per the sampling frequency.

Working Fluid Unit stores the reagents required to run the pAQ monitor for daily sampling. The modular nature of this unit facilitates easy maintenance and refilling of the reagents during regular building maintenance. The size of this unit can be increased or decreased based on the volume of reagent required, space availability, and ease of handling the reagent bottle while refilling the liquids.

Air Pump is externally housed, either in the open or concealed inside a soundproof location. The modular design allows for the air pump to be placed away from the pAQ monitor unit, this reduces the noise level exposure by the user.

The pAQ monitor duct air sampling unit can be operated unattended for weeks to up to a month (or more) with minimum human assistance.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that vary depending upon the desired properties sought to be obtained by a particular embodiment. In some embodiments, the numerical parameters are be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein.

In some embodiments, the terms “a” and “an” and “the” and similar references used in the context of describing a particular embodiment (especially in the context of certain of the following claims) are construed to cover both the singular and the plural, unless specifically noted otherwise. In some embodiments, the term “or” as used herein, including the claims, is used to mean “and/or” unless explicitly indicated to refer to alternatives only or to refer to the alternatives that are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and may also cover other unlisted steps. Similarly, any composition or device that “comprises,” “has” or “includes” one or more features is not limited to possessing only those one or more features and may cover other unlisted features.

All methods described herein are performed in any suitable order unless otherwise indicated herein or otherwise clearly contradicted by context. The use of any and all examples, or exemplary language (e.g. “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member is referred to and claimed individually or in any combination with other members of the group or other elements found herein. One or more members of a group are included in, or deleted from, a group for reasons of convenience or patentability. When any such inclusion or deletion occurs, the specification is herein deemed to contain the group as modified thus fulfilling the written description of all Markush groups used in the appended claims.

To facilitate the understanding of the embodiments described herein, a number of terms are defined below. The terms defined herein have meanings as commonly understood by a person of ordinary skill in the areas relevant to the present disclosure. Terms such as “a,” “an,” and “the” are not intended to refer to only a singular entity, but rather include the general class of which a specific example may be used for illustration. The terminology herein is used to describe specific embodiments of the disclosure, but their usage does not delimit the disclosure, except as outlined in the claims.

All of the compositions and/or methods disclosed and claimed herein may be made and/or executed without undue experimentation in light of the present disclosure. While the compositions and methods of this disclosure have been described in terms of the embodiments included herein, it will be apparent to those of ordinary skill in the art that variations may be applied to the compositions and/or methods and in the steps or in the sequence of steps of the method described herein without departing from the concept, spirit, and scope of the disclosure. All such similar substitutes and modifications apparent to those skilled in the art are deemed to be within the spirit, scope, and concept of the disclosure as defined by the appended claims.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. An airborne detection device for analyzing an environmental air sample and detecting airborne pathogens, the device comprising:
an analysis vial; and
a biosensor electrode.

2. The device of claim 1, wherein the device is configured to be used to detect multiple pathogens simultaneously in a single test.

3. The device of claim 2, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.

4. The device of claim 1, wherein the device is used to test for multiple variants of a pathogen from a single test.

5. The device of claim 4, wherein the multiple variants of a pathogen include delta and omicron variants of SARS-CoV-2.

6. The device of claim 1, wherein the device is configured to be used in multiplex tests.

7. The device of claim 6, wherein in the multiplex tests, multiple pathogens are detected simultaneously.

8. A method for detecting airborne pathogens, the method comprising:
transporting a liquid sample from an external sampling device to an analysis vial of an airborne detection device;
adding working fluid to the analysis vial of the airborne detection device;
detecting at least one pathogen;
replenishing sample fluid to the external sampling device; and
evacuating an analyzed sample from the analysis vial to a waste reservoir of the airborne detection device.

9. The method of claim 8, wherein detecting at least one pathogen comprises detecting multiple pathogens simultaneously in a single test.

10. The method of claim 9, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.

11. The method of claim 8, wherein detecting at least one pathogen comprises detecting multiple variants of a pathogen from a single test.

12. The method of claim 11, wherein the multiple variants of a pathogen include delta and omicron variants of SARS-CoV-2.

13. The method of claim 8, wherein detecting at least one pathogen comprises multiplex detection, wherein multiple pathogens are detected simultaneously.

14. A system for detecting airborne pathogens, the system comprising:
an airborne detection device; and
an external sampling device.

15. The system of claim 14, wherein the system is used to detect multiple pathogens simultaneously in a single test.

16. The system of claim 15, wherein the multiple pathogens are combinations of pathogens, wherein the pathogens are each selected from the group consisting of viruses, bacteria, parasites, fungi, mold, multiple viruses, multiple bacteria, multiple species or strains of viruses, multiple species or strains of bacteria, multiple species or strains of parasites, multiple species or strains of fungi, and multiple species or strains of mold.

17. The system of claim 14, wherein the system is used to test for multiple variants of a pathogen from a single test.

18. The system of claim 17, wherein the multiple variants of a pathogen include delta and omicron variants of SARS-CoV-2.

19. The system of claim 14, wherein the system is configured to be used in multiplex tests.

20. The system of claim 19, wherein in the multiplex tests, multiple pathogens are detected simultaneously.
